(19) United States
(12) Patent Application Publication
(10) Pub. No.: US 2022/0072081 A1
(43) Pub. Date: Mar. 10, 2022

(54) METHODS OF USING IRON-FREE SUPPLEMENTS TO ENHANCE CYTOPROTECTION AGAINST ANEMIA IN HUMANS

(71) Applicants: Bernard W. Downs, Lederach, PA (US); Steven M. Kushner, Oldsmar, FL (US)

(72) Inventors: Bernard W. Downs, Lederach, PA (US); Steven M. Kushner, Oldsmar, FL (US)

(21) Appl. No.: 17/239,142

(22) Filed: Apr. 23, 2021

Related U.S. Application Data
(60) Provisional application No. 63/014,427, filed on Apr. 23, 2020.

(51) Int. Cl. A61K 36/9068; A61K 9/127; A61K 47/24; A61K 36/481; A61K 36/70; A61K 36/82; A61K 36/28; A61K 36/738; A61K 36/23; A61K 36/254; A61K 36/734; A61K 36/185; A61K 36/15; A61K 36/886; A61K 31/07; A61K 31/375; A61K 31/593; A61K 31/355; A61P 7/06

(52) U.S. Cl. CPC A61K 36/9068; A61P 7/06; A61K 47/24; A61K 36/481; A61K 36/70; A61K 36/82; A61K 36/28; A61K 36/738; A61K 36/23; A61K 36/254; A61K 36/734; A61K 36/185; A61K 36/15; A61K 36/886; A61K 31/07; A61K 31/375; A61K 31/593; A61K 31/355; A61K 9/127

(57) ABSTRACT

A multi-nutrient containing formulation is provided which is encapsulated in phospholipid structures. The multi-nutrient containing formulation may be administered to human subjects to restore hemoglobin in red blood cells (RBCs) and restore white blood cells. The multi-nutrient containing formulation may be administered to human subjects to normalize platelet morphology in anemic subjects.

This application claims the benefit of U.S. Provisional Application No. 63/014,427, filed on Apr. 23, 2020, which is hereby incorporated by reference herein in its entirety.

### TECHNICAL FIELD

A multi-nutrient containing formulation is described which is encapsulated in phospholipid structures. The multi-nutrient containing formulation may be administered to human subjects to restore hemoglobin in red blood cells (RBCs) and restore white blood cells. The multi-nutrient containing formulation may be administered to human subjects to normalize platelet morphology in anemic subjects.

### BACKGROUND

Anemia is a health-deteriorating condition characterized by a lower-than-normal red blood cell count, or in which red blood cells do not have adequate hemoglobin to effectively carry sufficient oxygen to the body's vital organs and tissues. See, e.g., N. J. Kassebaum, et al., The Global Burden of Anemia, 30 HEMATOLOGY/ONCOLOGY CLINICS OF N. AM. 247 (2016); MAYO CLINIC, Iron deficiency anemia, MAYO CLINIC.ORG (Nov. 11, 2016), https://www.mayoclinic.org/diseases-conditions/iron-deficiency-anemia/symptoms-causes/syc-20355034; N. J. Kassebaum, et al., A systematic analysis of global anemia burden from 1990 to 2010, 123 BLOOD 615 (2014); Clara Camaschella, Iron-Deficiency Anemia, 372 NEW ENGLAND J. MEDICINE 1832 (2015); S. Killip, et al., Iron deficiency anemia, 75 AM. FAMILY PHYSICIAN 671 (2007); each of which is incorporated by reference herein in its entirety. Thus, deficiency of oxygen-rich red blood cells is the prime indicator of anemia, which may occur in both men and women of any age group temporarily or more persistently. See, e.g., M. A. López & F. C. Martos, Iron availability: An updated review, 55 INT'L J. FOOD SCIS. & NUTRITION 597 (2004); B. Lozoff, et al., Long-term developmental outcome of infants with iron deficiency, 325 NEW ENGLAND J. MEDICINE 687 (1991); S. R. Lynch, Why nutritional iron deficiency persists as a worldwide problem, 141 J. NUTRITION 763S (2011); each of which is incorporated by reference herein in its entirety. However, women of childbearing age are more prone to anemia, and are especially prone during pregnancy. Anemia can range in degree from mild to severe. Signs and symptoms of anemia can include, but are not limited to, fatigue or tiredness, weakness, exhaustion, pale or yellowish skin, irregular heartbeats, shortness of breath, dizziness or lightheadedness, chest pain, cold hands and feet, and/or headache. See, e.g., Lorna G. Macdougall, et al., Red cell metabolism in iron deficiency anemia. II. The relationship between red cell survival and alterations in red cell metabolism, 76 J. PEDIATRICS 660 (1970); J. L Miller, Iron deficiency anemia: a common and curable disease, 3 COLD SPRING HARBOR PERSPECTIVES IN MEDICINE a011866 (2013); P. G. Fraenkel, Anemia of Inflammation: A Review, 101 MEDICAL CLINICS OF N. AM. 285 (2017); each of which is incorporated by reference herein in its entirety.

There are five main known causes of anemia: (a) blood loss due to excessive menstrual bleeding or bleeding in the digestive or urinary tract, surgery, trauma, or cancer; (b) substantial loss of blood leading to significant alterations in the blood parameters including reduced iron; (c) lack of ability to produce red blood cells; (d) inherited compromised rate of red blood cell production; and (e) chemotherapy or radiation therapy. There are four major categories of iron-containing proteins present in the human body: (i) mononuclear proteins; (ii) di-iron-carboxylate proteins; (iii) iron-sulfur proteins; and (iv) heme proteins. Among the four categories, heme proteins are the most abundant in the human body. Notably, hemoglobin, which is located in erythrocytes, contains approximately 50% of the total iron in the body. Accordingly, lack of hemoglobin plays a central role in the etiology of anemia. Erythropoiesis, the process for the production of erythrocytes, is regulated by three prime factors: (a) appropriate oxygenation of tissues and organs; (b) turnover of erythrocytes; and (c) loss of erythrocytes due to hemorrhage. See, e.g., Mitchell H. Rosner & Mark A. Perazella, Acute Kidney Injury in Patients With Cancer, 32 ONCOLOGY 351 (2018); Mitchell H. Rosner & Mark A. Perazella, Acute Kidney Injury in Patients With Cancer, 376 NEW ENGLAND J. MEDICINE 1770 (2017); K. Nishikura, Identification of histidine-122a in human haemoglobin as one of the unknown alkaline Bohr groups by hydrogen-tritium exchange, 173 BIOCHEMICAL J. 651 (1978); each of which is incorporated by reference herein in its entirety.

Erythrocytes and their precursors, erythroblasts, need a requisite amount of functional iron and oxygen for the production of hemoglobin and heme. It is well known that iron is centrally located in the hemoglobin structure, and is essential for hemoglobin function. It is well established that erythroblasts are nucleated cells found in the bone marrow and are the precursors of erythrocytes. Mono- and di-ferric transferrin, present in considerable amounts in plasma, are the immediate sources of functional iron for erythroblasts. Generally, anemia is associated with low iron function in available transferrin. Iron is available from three major physiological sources: (a) gut; (b) macrophages; and (c) hepatic tissues, which store ferritin iron. Generally, iron storage declines or is depleted before anemia develops. Hence, dietary and rejuvenated iron have been considered the primary necessity for erythrocyte production. Absent hemorrhage or disease conditions, tissue oxygenation and erythrocyte production are stable during adulthood. During hemorrhage, oxygen and iron availability dramatically decline, which may lead to anemia and fatigue. Moreover, anemia is intricately linked to chronic inflammation, chronic kidney disease, gastrointestinal and gynecological malignancies, and autoimmune disease.

Chronic anemia syndrome (“CAS”), as used herein, alone or in combination with other terms, unless stated otherwise, refers to all forms of iron deficiency anemia (“IDA”) that are not caused by genetics or hemorrhage, and includes chronic inflammatory disorders induced by an increasingly anaerobic/acidic environment, thereby promoting the growth of anaerobic organisms. Chronic anemia syndrome (CAS) is characterized by a deficiency of primary intracellular alkalinizing ion buffers, which induces a defensive expenditure of secondary alkaline buffers from hemoglobin (i.e., histidine), in order to prevent catastrophic decrease in blood pH. It is well known that a heme group is attached to four histidines, while the iron atom in heme binds to the four nitrogens at the center of the protoporphyrin ring. See, e.g., Márcio Simao, et al., Iron-enriched diet contributes to early onset of osteoporotic phenotype in a mouse model of hereditary hemochromatosis, 13 PLOS ONE e0207441 (2018), incorporated by reference herein in its entirety. Chronic anemia syndrome (CAS) causes the cleavage of iron from the heme protein to release histidine. Subsequently, iron is initially taken out of circulation and is stored in hepatic and other tissues, which leads to excessive accumulation of iron in the organs and, ultimately, hemochromatosis, which is a pathophysiological condition known as “iron overload anemia.” See, e.g., P. C. Adams, Epidemiology and diagnostic testing for hemochromatosis and iron overload, 37 (Suppl. 1) INT'L J. LAB. HEMATOLOGY 25 (2015), incorporated by reference herein in its entirety. In severe hemochromatosis, iron saturation in the organs results in a down-regulation or cessation of deposition in those organs, causing excessive accumulation of iron in blood plasma. See, e.g., H. Zoller & B. Henninger, Pathogenesis, Diagnosis and Treatment of Hemochromatosis, 34 DIGESTIVE DISEASES 364 (2016), incorporated by reference herein in its entirety. Red blood cells are unable to restore hemoglobin status due to the excess expenditure of histidine from heme proteins required to maintain ideal alkaline blood pH properties and oxygen utilization. Accordingly, chronic anemia syndrome (CAS) refers to the progressive inability of the human body to effectively use cellular oxygen, which in turn induces a progressive acidemia in the blood supply, as evidenced by an increasingly hypochromic state due to a significant loss of hemoglobin. This cascade of physiological conditions forces a metabolic shift toward cellular anaerobic glycolysis, and a compensatory expenditure of alkalinizing histidine molecules from the heme protein of deconjugated hemoglobin, which releases iron.

The efficacy of oral administration of a novel and safe iron free VMP35 MNC encapsulated in a proprietary SK713 SLP phospholipid Prodosome technology on anemia and blood properties (i.e., hemoglobin) was assessed in human volunteers. See, e.g., B. W. Downs, et al., the effect tof VMP35 supplement ingredients encapsulated in a novel phospholipid Prodosome SK713 SLP nutrient delivery technology observed as a result of changes in properties of live human blood, 5 FUNCTIONAL FOODS IN HEALTH & DISEASE 292 (2015), incorporated by reference herein in its entirety. See also US Pat. Appl. Publ. No. 2017/0049701 to Kushner et al., incorporated by reference herein in its entirety. However, the etiology of what has been believed to be “iron deficiency anemia” has been heretofore misconceptualized and therefore misunderstood. Because the actual etiology has only now been discovered as a result of a breakthrough paradigm shift, there exists a long-felt need for new treatment regiments for anemic disorders and related pathologies embraced within the term chronic anemia syndrome (CAS). Consequently, further research and development is required to develop treatment regimens for the set of interrelated anemic disorders and related pathologies known as chronic anemia syndrome (CAS) and caused by the induction of hemoglobin expenditure as an alkaline buffer.

It is well-known that phospholipids are important molecules in biological systems. Cells are surrounded by a layer of phospholipids called the phospholipid bilayer (generally, “lipid bilayer”). This layer makes up your cellular and intracellular organelle membranes, forming a selectively permeable barrier, and is critical to a cell's ability to function. Phospholipids are arranged so that their water-repelling (hydrophobic) or “fat-loving” tails are pointing inwards and their water-attracting (hydrophilic) heads are pointing outwards in this bilayer structure. This arrangement allows plasma membranes to be selectively permeable to dissolved substances such as proteins, ions, and water. In biological systems, phospholipids allow cell membranes to be fluid. Their unique characteristics allow the cell membrane to be more malleable, taking different shapes and expanding or shrinking when necessary, such as when cells have to travel through very narrow capillaries in single file, one at a time. Phospholipids also can act as signaling molecules for receptors inside and outside of cell surfaces, facilitating communications between cells. They can be split to produce secondary messengers in cellular systems. As a secondary messenger, phospholipids can signal for leukocytes to migrate to a site of infection, and they can also inhibit nerve cells when necessary.

Important Functions of Phospholipids

(1) Act as building blocks of the biological cell membranes in virtually all organisms.

(2) Participate in the transduction of biological signals across cell membranes.

(3) Act as efficient store of energy as with triglycerides.

(4) Play an important role in the transport of fat between gut and liver in mammalian digestion.

(5) Serve as an important source of acetylcholine which is the most commonly occurring neurotransmitter substance occurring in mammals.

One of the outcomes of a healthy diet combined with healthy digestion is the formation of liposomes from phospholipids in the diet. Owing to the diminished quality of the standard American diet, and the consequential wide-spread decline of digestive competence, the formation of liposomes in the gastrointestinal (“GI”) tract has been significantly compromised and diminished. Without the aid of the liposome, many of the nutrients would not otherwise adequately penetrate the epithelial wall of the intestines for eventual uptake into the cells. Liposomes are safe and important for facilitating optimal absorption of valuable nutrients. For example, naturally occurring liposomes are present in human breast milk. See, e.g., M. M. Koerner, et al., Electrodynamics of lipid membrane interactions in the presence of zwitterionic buffers, 101 BIOPHYSICAL J. 362 (2011), incorporated by reference here in its entirety. Liposome structures are biodegradable and biocompatible (“body friendly”), enabling absorption through most tissues in the GI tract and alimentary tract, from the mouth to the colon. In addition to water-soluble vitamins, liposomes are beneficial for effective in situ delivery of fat-soluble vitamins, trace minerals, and naturally occurring phytonutrients, including flavonoids, terpenes, and saponins. See, e.g., B. C. Keller, Liposomes in nutrition, 12 TRENDS IN FOOD SCI. & TECH. 25 (2001), incorporated by reference herein in its entirety.

If new methods of treating chronic anemia syndrome in humans with iron-free supplements could be developed, this would represent a useful contribution to the art.

### SUMMARY OF THE INVENTION

Prodovitex is easily and rapidly absorbed via the sublingual mucosa (and then again through the GI mucosal border), gaining rapid (and sustained) access to the blood, restoring hemoglobin and white blood cells and normalizing platelet morphology in anemic patients. Prodovite® is also beneficial as a daily nutritional supplement containing vitamins, minerals, and phytonutrients. As a result of restoring hemoglobin, Prodovite® improves oxygen, water, and nutrient availability and utilization, enhancing the ability for cells to make energy, manage energy and eliminate wastes; enhancing ‘cellular metabolism’, all which cannot be done effectively by anemic red blood cells.

In one embodiment, a method for treating or preventing chronic anemia syndrome in a human subject in need of such treatment is described, comprising the steps of (a) providing a formulation containing one or more vitamins or multivitamins, one or more macro or trace minerals, one or more botanical nutrients or phytonutrients, and a multilamellar clustoidal phospholipid vehicle, the multilamellar clustoidal phospholipid vehicle comprising: a solvent, phosphatidylcholine of at least 70% purity, and an ionic mineral composition; (b) administering the formulation in an effective amount to treat the human subject; and measuring at least one blood parameter in the human subject. In an alternative embodiment, a method for improving physical performance is contemplated using the formulation.

In an embodiment, red blood cell (RBC) morphology and hemoglobin saturation are improved after administration of said formulation.

In an embodiment, the formulation can be administered orally or tranmucosally.

### BRIEF DESCRIPTION OF THE DRAWINGS

FIG. 1 depicts, in one embodiment, a representative example from Group 1 (Subject #45) via transmucosal delivery, human subjects: (a) Baseline before intake of water; (b) Five minutes after taking 30 mL water; and (c) Five minutes after taking 30 mL VMP35 MNC.

FIG. 2 depicts, in one embodiment, a representative example from Group 2 (Subject #10): (a) Baseline blood test before the administration of VMP35 MNC; and (b) Five minutes after taking VMP35 MNC.

FIG. 3 depicts, in one embodiment, a representative example from Group 2 (Subject #11): (a) Baseline blood test before the administration of 30 mL VMP35 MNC; and (b) Five minutes after taking VMP35 MNC.

FIG. 4 depicts, in one embodiment, a representative example from Group 2 (Subject #40): (a) Baseline blood test before the administration of 30 mL VMP35 MNC; and (b) Five minutes after taking VMP35 MNC.

FIG. 5 depicts, in one embodiment, a representative example from Group 3 (Subject #49): (a) Baseline blood test before the administration of VMP35 MNC; (b) Five minutes after taking 30 mL VMP35 MNC; and (c) Thirty minutes after taking 30 mL VMP35 MNC.

FIG. 6 depicts, in one embodiment, a representative example from Group 3 (Subject #49): (a) Baseline blood test before the administration of VMP35 MNC; (b) Five minutes after taking 30 mL VMP35 MNC; and (c) Thirty minutes after taking 30 mL VMP35 MNC.

FIG. 7 depicts, in one embodiment, live blood cell imaging of a representative example from Group 1 (Subject #41) at Baseline before intake of water by oral delivery.

FIG. 8 depicts, in one embodiment, live blood cell imaging of a representative example from Group 1 (Subject #41) at five minutes after intake of water by oral delivery.

FIG. 9 depicts, in one embodiment, live blood cell imaging of a representative example from Group 1 (Subject #41) at five minutes after intake of Prodovite® VMP35 MNC by oral delivery.

FIG. 10 depicts, in one embodiment, live blood cell imaging of a representative example from Group 2 (Subject #17) at Baseline before intake of Prodovite® VMP35 MNC by oral delivery.

FIG. 11 depicts, in one embodiment, live blood cell imaging of a representative example from Group 2 (Subject #17) at five minutes after intake of Prodovite® VMP35 MNC by oral delivery.

FIG. 12 depicts, in one embodiment, live blood cell imaging of a representative example from Group 3 (Subject #45) at Baseline before intake of Prodovite® VMP35 MNC by oral delivery.

FIG. 13 depicts, in one embodiment, live blood cell imaging of a representative example from Group 3 (Subject #45) at five minutes after intake of Prodovite® VMP35 MNC by oral delivery.

FIG. 14 depicts, in one embodiment, live blood cell imaging of a representative example from Group 3 (Subject #45) at thirty minutes after intake of Prodovite® VMP35 MNC by oral delivery.

FIG. 15 shows, in one embodiment, components of the trace mineral concentrate.

### DETAILED DESCRIPTION

Prodovite® and Prodovite® VMP35 Multi-Nutrient-Complex (MNC), available from Victory Nutrition International, Inc. (Lederach, Pa.) is produced by first engineering a unique electrolytic enhanced non-GMO high potency phosphatidylcholine rich transport sphere. Then, a highly specific blend of whole food extracts and nutrient isolates are processed with a unique high sheer wet milling process to solubilize these ingredients creating a nano-emulsion. Then, the ingredients are mixed using a precise sequential folding method to ensure all nutrients are “captured” within the phospholipid transport spheres, known as Prodosomes. As the emulsified ingredients are added to the phospholipid substrate, it creates many levels of ionically-enhanced phospholipid spheres (aka a ‘multilamellar clustoidal architecture’) containing the pre-digested nano-emulsified ingredients. Any deviation in this sequence of production results in an unsuccessful material.

As used herein, the term “iron-free” means that in certain embodiments the formulations are essentially free of iron, or alternatively, contain only trace amounts of residual iron as determined by standard analytical techniques such as ICP or ICP-MS.

As used herein, the term “clustoid(s),” alone or in combination with other terms, unless stated otherwise, refers to clusters of liposomal spheres.

As used herein, the term “multilamellar clustoidal,” alone or in combination with other terms, unless stated otherwise, refers to clusters of liposomal spheres within a liposomal sphere and clusters of those liposomal spheres within a liposomal sphere, etc., up to hundreds of concentric layers.

As used herein, the term “prodosome,” alone or in combination with other terms, unless stated otherwise, refers to the “energetically enhanced (EFlquence-treated) liposome that comprises the complex of multilamellar clustoidal liposomal structures.” Specifically, “prodosome” refers to electrolyte (ion)-impregnated phospholipid liposome complexes having multilamellar clustoidal liposomal structures.

As used herein, the term “liposome,” alone or in combination with other terms, unless stated otherwise, refers to a vesicle composed of amphiphilic lipids arranged in a spherical bilayer or bilayers. Liposomes are unilamellar or multilamellar vesicles that have a membrane formed from a lipophilic material and an aqueous interior that contains the composition to be delivered. In order to cross intact mammalian skin, lipid vesicles must pass through a series of fine pores, each with a diameter less than 50 nanometers, under the influence of a suitable transdermal gradient. Therefore, it is desirable to use a liposome that is highly deformable and able to pass through such fine pores.

As used herein, the term “bioavailability,” alone or in combination with other terms, unless stated otherwise, refers to a measurement of that portion of an administered drug that reaches the circulatory system (e.g., blood, especially blood plasma) when a particular mode of administration is used to deliver the drug. Enhanced bioavailability refers to a particular mode of administration's ability to deliver nutrients, including oligonucleotides, nutraceutical particles, and drugs to the peripheral blood plasma of a subject tin need relative to another mode of administration. For example, when a non-parenteral mode of administration (e.g., an oral mode) is used to introduce the drug into a subject in need, the bioavailability for that mode of administration may be compared to a different mode of administration. Further, bioavailability correlates with therapeutic efficacy when a compound's therapeutic efficacy is related to the blood concentration achieved.

As used herein, the term “chronic anemia syndrome (‘CAS’),” alone or in combination with other terms, unless stated otherwise, refers to all forms of iron deficiency anemia (“IDA”) that are not caused by genetics or hemorrhage, and includes chronic inflammatory disorders induced by an increasingly anaerobic/acidic/hypoxic environment, thereby enabling the growth of anaerobic organisms. Chronic anemia syndrome (CAS) is characterized by a deficiency of primary intracellular alkalinizing ion buffers, which induces a defensive expenditure of secondary alkaline buffers from hemoglobin (i.e., histidine), in order to prevent catastrophic decrease in blood pH. Chronic anemia syndrome (CAS) causes the cleavage of iron from the heme protein to release histidine. Subsequently, iron is initially taken out of circulation and is stored in hepatic and other tissues, which leads to excessive accumulation of iron in the organs and, ultimately, hemochromatosis, which is a pathophysiological condition known as “iron overload anemia.” See, e.g., P. C. Adams, Epidemiology and diagnostic testing for hemochromatosis and iron overload, 37 (Suppl. 1) INT'L J. LAB. HEMATOLOGY 25 (2015), incorporated by reference herein in its entirety. In severe hemochromatosis, iron saturation in the organs results in a down-regulation or cessation of deposition in those organs, causing excessive accumulation of iron in blood plasma. See, e.g., H. Zoller & B. Henninger, Pathogenesis, Diagnosis and Treatment of Hemochromatosis, 34 DIGESTIVE DISEASES 364 (2016), incorporated by reference herein in its entirety. Red blood cells are unable to restore hemoglobin status due to the excess expenditure of histidine from heme proteins required to maintain ideal alkaline blood pH properties and oxygen utilization. Accordingly, chronic anemia syndrome (CAS) refers to the progressive inability of the human body to effectively use cellular oxygen, which in turn induces a progressive acidemia in the blood supply, as evidenced by an increasingly hypochromic state due to a significant loss of hemoglobin. This cascade of physiological conditions forces a metabolic shift toward cellular anaerobic glycolysis, and a compensatory expenditure of alkalinizing histidine molecules from the heme protein of deconjugated hemoglobin, which releases iron.

Prodosomes

While the liposome is naturally a zwitterionic molecule, the inclusion of the mineral ions in a similar proportion that exists in human blood, within every portion of the present complex of liposomal clustoidal spheres creates previously non-existent electrical properties of the liposomes (called “prodosomes”). Based on electrostatic properties, mineral ions incorporated into the water used for creating liposomes become part of the liposome structure itself; resembling the ionic properties that exist in human blood, for example. This enhances the ability of the liposomal transport sphere to transport and facilitate encapsulated nutrient absorption. This is in addition to encapsulating supplemental minerals of a nutrient formula containing one or more nutrient components within their liposomal spherical structures as a nutrient or nutritional payload.

In one embodiment, the solar-dried electrolyte source material being infused into the phospholipids is ionic in nature. This property infuses the ions into the manufactured liposomes and creates electrical/energetic/frequency properties of the phospholipid-based liposomal structures. The liposome (prodosome), in essence, becomes a dynamically charged compound, resembling more of a biological material, with similar ionic amounts as exist in human blood, with greater bio-functionality and potential for transport and delivery of nutrients, and contributing beneficial biological activities on their own.

Electrolytes are also important intracellular pH buffers. Following the depletion of intracellular electrolytes and exhaustion of other primary buffers, hemoglobin is expended to maintain intracellular pH. This changes not only the oxygen-carrying abilities of the hemoglobin but also polarity (negative ion concentration), and results in excessive red blood cell aggregation. Improvement in red blood cell morphology and plasma rheology, for example, are evidence of improvements in blood viscosity, negative ion concentration, pH, and blood functionality, i.e., oxygenating and hydrating properties.

With the liposome (prodosome) now infused and saturated with a comprehensive range of naturally occurring energetically active ions, there is a greater potential that the entire multilamellar clustoidal structure may act as a pH buffering agent for the tissues. It is likely that there is a re-balancing of pH in tissues where the liposome releases its payload as well as when the liposomal membranes sequentially begin to degrade and release their bioactive ions. This re-balancing of pH and restoration of optimal ionic properties will foster a more advantageous environment for nutrient utilization. As pH rebalances, healthy blood morphology, rheology, and hematology (i.e., viscosity, form, structure, oxygenation, hydration, etc.) are restored.

Phospholipids have an adhesive property owing to the hydrophilic and hydrophobic properties of the molecule. As a result, a natural tendency of a phosphatidyl choline-based liposome is its ability to adhere to tissues, especially the mucosa of the GI tract. This attribute promotes transmucosal nutrient transport from the sublingual tissues in the mouth to the tissues of the intestine. Prolonged adherence of the liposome to the surface of the villi and microvilli translates to a longer portion of time that nutrients can diffuse across the membranes into the blood stream. More importantly, the extended time that the liposome remains attached to the mucosal membrane gives additional time for the mineral ions to saturate the same membranes. Moreover, the lipid bilayer construction created by phospholipids is readily incorporated into the cell membrane phospholipid bilayer. By continually saturating the junctions where nutrients are absorbed, an advantage is afforded for more complete nutrient transport. This is due to mineral ions' contribution to maintaining the osmotic gradient in the lipid bilayer of cell membranes that facilitate nutrient diffusion and maintain electroneutrality.

The fact that the liposome remains attached to the mucous membrane for longer periods of time means the mineral ions remain there as well. Again, this means that there is a longer period of time where nutrient exchange, facilitated by cellular ions, can be carried out. Moreover, the rationale of infusing the mineral ions within the entirety of the liposome is borne out by the fact that as each layer of the multi-lamellar sphere degrades and releases its nutrients in the GI mucosa, there is a simultaneous and consistent release of mineral ions as a result of liposomal (prodosome) degradation. This is as opposed to bound minerals just being present within the sphere of a “simple” liposome that can release at a single instant and then must be absorbed into the bloodstream. In an embodiment, the process of the present disclosure enables mineral ions to be available throughout the entire process where each successive layer of phospholipids and their nutrient contents, both fat and water-soluble, are being released from the disintegrating spheres, along with phospholipid-infused ions, and made available for diffusion and bioactivity.

Without being bound by theory, in an embodiment, the present invention contemplates the ability to increase Zeta Potential within the liposome (prodosome) itself and consequently in surrounding fluids where the liposome degrades and releases its nutritional payload, including its free ions. As used herein, the term “Zeta Potential,” alone or in combination with other terms, unless otherwise stated, refers to the electrical potential of dispersed particles in colloidal solutions. The higher the Zeta Potential, the greater the dispersion and subsequent stability of the solution. A higher Zeta Potential indicates a stronger level of electrostatic repulsion within the solution, and consequently, a more stable liposome, which is understood to be a key factor in maintaining the biologically active properties and efficacy of a nutritional compound or nutrient formula. This not only holds true for the solution (i.e., the liposomal concentrate), but this potential can also be transferred to the surrounding tissues as the liposome disintegrates/degrades. The electrostatic repulsion and separation of biological materials (i.e., erythrocytes, leukocytes, platelets, etc.) is exactly the environment that is desirable within the bloodstream of a human subject, for example.

Without being bound by theory, in an embodiment, the electrostatic repulsion and separation of biological materials (i.e., erythrocytes, leukocytes, platelets, etc.) helps to ensure adequate red blood cell circulation and, therefore, oxygenation, over a larger surface area. The opposite consequence (i.e., inadequate red blood cell circulation and oxygenation over a given surface area) would be aggregation and less free-flowing red blood cells. Accordingly, in an aspect, the present invention includes, but is not limited to, the creating of a transfer of Zeta Potential through the direct action of the liposome itself as it enters into surrounding plasma. As the Zeta Potential increases in the surrounding blood, allowing for better circulation of red blood cells, the overall rheology of the blood is improved, thereby allowing for a greater flow of the nutrient payload that has been delivered by the multilamellar clustoidal liposome structures. Research has shown that both Zeta Potential and particle size within a colloidal solution can be modified by the inclusion of an ionic species, for example, according to embodiments of the presently disclosed clustoidal multilamellar SLP structures (prodosomes).

Without being bound by theory, recent research has also shown that varying degrees of vortex speed can decrease particle size in a colloidal solution while simultaneously increasing Zeta Potential, while also serving to allow embodiments of the present invention to increase surface area coverage. Embodiments of the present invention can increase surface area coverage through the use of high-speed RPMs within small mixing containers, allowing the mineral ions to more thoroughly disperse in a more uniform manner within the phospholipid matrix, which directly leads to a higher Zeta Potential. Moreover, typical Zeta Potential has to do with an electrokinetic potential between the surface of the colloidal particle and any point in the mass of the liquid medium. Without being bound by theory, it is expected that because embodiments of the present invention involve increasing ionic concentrations within the water prior to the production of the multi-lamellar liposome, multiple surfaces are generated, surrounded by multiple liquid mediums, into which the active substrate can permeate the inter-phospholipid molecular spaces or interstitial lumens. Thereby, in embodiments of the present invention, the multi-lamellar SLP structure is produced. Additionally, according to embodiments of the present invention, a Zeta Potential has been created within the multitude of layers of a clustered multi-lamellar liposomal sphere (i.e., prodosome). Consequently, in embodiments of the present invention, as the liposome dissolves sequentially layer by layer, positive benefits of the increased Zeta Potential from each surface layer is conferred into the surrounding medium into which the liposome dissolves, specifically, the sublingual mucosa (alimentary) and small intestine (GI), facilitating rapid and prolonged absorption into the bloodstream.

The absorption of food and most supplemental minerals primarily takes place within the small intestines, although ionic minerals can be absorbed through the sublingual mucosa. As food matter passes through the intestines, minerals transfer into the blood stream through the walls of the intestines by way of the villi. Without being bound by theory, minerals transferring into the blood stream by way of the intestinal villi can only happen if the minerals are in an ionic form. When the stomach is functioning properly, stomach acid normally ionizes minerals in foods and supplements. However, according to statistics, properly functioning stomachs are not commonplace in North America. Most mineral supplements contain bonded minerals (e.g., calcium carbonate, magnesium oxide, etc.) that must be ionized for optimal absorption and utilization in the body.

In one aspect of the present invention, encapsulating nutritional, nutraceutical, or pharmaceutical substrate(s) in soy lecithin phospholipid (“SLP”) capsules enables superior absorption of nutritionally and pharmacologically therapeutic substances. The present disclosure offers significant therapeutic health benefits due to its energetically enhanced phospholipid properties impacting delivery of nutrients and/or drugs, including, but not limited to: (1) neuroprotection, regulation of brain activity, improved memory and resistance to stress, reduced depression risk, and mitigation of the progression of neurodegenerative diseases like ALS, MS, Alzheimer's disease, and Parkinson's disease; (2) positive influences on cellular growth, development, and energy generation due to participation in molecular transport, and cellular organelle and intracellular organelle structure and function; (3) acceleration of tissue and organism regeneration after trauma, damage, illness, and/or physical exertion, including wound healing; (4) limiting cholesterol absorption from the gastrointestinal tract; (5) beneficial outcomes in liver therapy (e.g., steatosis, alcohol intoxication, etc.); (6) inhibition of inflammation factors, some of which are pathogens of the alimentary canal and cancer promoters (e.g., of colon and adenoma); and (7) immune support. See, e.g., B. C. Keller, 2001.

More specifically, in certain embodiments, the present disclosure is directed to a clustoidal multi-lamellar phospholipid based material (“prodosome”) that is infused and fortified with an electrolyte mineral complex including more than 70 naturally occurring macro- and trace minerals in ionic form. As used herein, the term “trace minerals,” alone or in combination with other terms, unless stated otherwise, refers to naturally occurring minerals derived from, for example, evaporated inland sea water in an ionic form. Trace minerals, include, but are not limited to, iron ion, copper ion, zinc ion, manganese ion, selenium ion, chromium ion, iodine ion, and boron ion. Macro minerals include, but are not limited to, calcium ion, magnesium ion, phosphorous ion, potassium ion, chloride ion, and sulfur ion. In embodiments of the present invention, the final material possesses an electrical potential structurally integrated into the SLP sphere at a micron/nano level. The SLP sphere, according to embodiments of the present invention, has now become more than just a transport compartment, but also possesses its own unexpected beneficial functionality facilitating improved utilization of nutrients encapsulated within the SLP liposomal spheres. In certain embodiments, the present invention provides a myriad of electrolytic materials, simultaneously with encapsulated nutrients, that contribute to and govern cellular fluid balance and, therefore, are instrumental in all metabolic processes including cellular exchange of nutrients and waste removal.

Existing liposome technologies use mostly lecithin and, especially those of higher quality, phosphatidyl choline. Regardless of the phospholipid source material, existing technologies are generally mixed in a stereotypical fashion with no other additives or compounds utilized within the source material(s). The result is generally a relatively unstable product that degrades of its own accord in a relatively short period of time due to variations in: temperature; agitation; composition of the substrate; interaction of the phospholipids with the encapsulated substrate; and pH; among other factors. The relative instability results in agglomeration leading to degradation and delineation of the phospholipid bilayer membrane of the multilamellar spheres.

Biological Capacitor

In embodiments of the present invention, the prodosome technology as described herein creates clusters of multilamellar liposomal structures in concentric layers of activated ion-infused liposomes within a liposome; and the multilamellar clusters of those molecules within an activated ion-infused liposome; up to hundreds of concentric layers, described as multilamellar liposomal clustoids, referred to herein as SK713 SLP Prodosomes, or “Prodosomes.” In addition to protecting the nutritional contents, complex multilamellar clustoidal structures (i.e., the “SK713 SLP” complex) effectively function as biological capacitors, containing and confining the biochemical and/or energetic potential of the ion-infused (energy frequency imprinting) phospholipids. However, this biological capacitor function would not occur in normal liposomes (see, e.g., Table 1) and is only possible because of the energy frequency imprinting (also known as “EFlquence” technology, available from Victory Nutrition International, Lederach, Pennsylvania, United States) of the SK713 SLP process technology.

One objective, in an embodiment of the present invention, is to supply already naturally ionized minerals that can be fully absorbed in vivo. The Energy Frequency Imprinting (trading as EFIquence™ Technology) process infuses and saturates the phospholipids with a full spectrum of solar-dried ionic minerals from ancient sea beds that supply minerals in biocompatible amounts and a proportion to the blood.

The electrolytes within the phospholipid matrix, according to embodiments of the present invention, are in ionic form, in which the electrolytes are in the most natural state, in which they are naturally charged, biologically active minerals that are bioavailable and soluble in water. This material is derived from the Great Salt Lake, then solar-dried, and contains over 72 ionic minerals that are about eight to about ten times more concentrated than regular seawater and significantly more concentrated than colloidal minerals. Colloidal minerals are of larger particles size, and contain no ionic charge, as compared to the trace minerals used in embodiments of the present invention. Additionally, the ions contained in the Prodosomes are at a similar percentage volume to that which is present in healthy human blood.

As described herein, in embodiments of the present invention, the biological capacitor function of the multilamellar SLP clustoids (Table 2) would not occur in normal liposomes (Table 1) and is only possible because of the energy frequency imprinting (also known as “EFIquence™” technology, available from Victory Nutritional International, Lederach, Pennsylvania, United States) of the SK713 SLP process technology. The novel multilamellar clustoidal phospholipid encapsulation technology of the present invention (SK713 SLP/“Prodosomes”) was developed to facilitate more stable, competent, and comprehensive synchronized absorption and synchronized bioavailability and bioactivity of orally ingested nutrition. The SK713 SLP is distinctly unique and superior to any previous liposomal technologies and, unlike previous versions: contains more phospholipid substrate, which is impregnated and saturated with solar dried electrolytes in an ionic state; is demonstrably and significantly more stable; and is consistently more uniform and shown to be more efficacious for nutrient delivery than other liposomal technologies tested. Moreover, the ion-infused SK713 SLP makes a nutritional contribution to improving the structure and function of inter- and intracellular membranes and molecules.

TABLE 1
Electrical Resistance of Normal Liposome Solutions (Reference)

| Sample #1 Pure Liposome | 1000X Setting (in Ohms) |
|---|---|
| Distilled Water = 50 ml. | 600 |
| Drops 1 | 400 |
| Drops 2 | 380 |
| Drops 3 | 380 |
| Drops 4 | 360 |
| Drops 5 | 350 |
| Drops 6 | 300 |
| Drops 7 | 300 |
| Drops 8 | 300 |
| Drops 9 | 300 |
| Drops 10 | 280 |
| Drops 11 | 280 |
| Drops 12 | 280 |
| Drops 13 | 280 |
| Drops 14 | 280 |
| Drops 15 | 280 |
| Drops 16 | 280 |
| Drops 17 | 280 |
| Drops 18 | 280 |
| Drops 19 | 260 |
| Drops 20 | 260 |
| Drops 21 | 260 |
| Drops 22 | 260 |
| Drops 23 | 260 |
| Drops 24 | 240 |
| Drops 25 | 240 |
| Drops 26 | 220 |
| Drops 27 | 220 |
| Drops 28 | 220 |
| Drops 29 | 220 |
| Drops 30 | 200 |
| Drops 40 | 160 |
| Drops 50 | 150 |
| Drops 60 | 150 |
| Drops 70 | 140 |
| Drops 80 | 135 |
| Drops 90 | 125 |
| Drops 100 | 115 |

TABLE2
Biological Capacitor Function of the Multilamellar SLP Clustoids

| Sample #2 | 1000X | 100X | 1000X | 100X |
|---|---|---|---|---|
| Prodosome (Multi- | Setting | Setting | Setting | Setting |
| lamellar SLP) | (in Ohms) | (in Ohms) | (in Ohms) | (in Ohms) |
| Distilled Water = | 600 | | 600 | |
| 50 mL | | | | |
| Drops 1 | 350 | | 350 | |
| Drops 2 | 220 | | 220 | |
| Drops 3 | 160 | | 160 | |
| Drops 4 | 140 | | 150 | |
| Drops 5 | 140 | | 140 | |
| Drops 6 | 120 | | 120 | |
| Drops 7 | 120 | | 120 | |
| Drops 8 | 120 | | 100 | |
| Drops 9 | 100 | | 90 | |
| Drops 10 | 90 | | 80 | |
| Drops 11 | 80 | | 80 | |
| Drops 12 | 80 | | 70 | |
| Drops 13 | 70 | | 66 | |
| Drops 14 | 70 | | 64 | |
| Drops 15 | 65 | | 62 | |
| Drops 16 | 65 | | 58 | |
| Drops 17 | 60 | | 56 | |
| Drops 18 | 60 | | 56 | |
| Drops 19 | 56 | | 54 | |
| Drops 20 | 54 | | 54 | |
| Drops 21 | 54 | | 52 | |
| Drops 22 | 52 | | 50 | |
| Drops 23 | 50 | | 50 | |
| Drops 24 | 50 | | 48 | |
| Drops 25 | 50 | | 48 | |
| Drops 26 | 48 | | 48 | |
| Drops 27 | 48 | | 46 | |
| Drops 28 | 46 | | 44 | |
| Drops 29 | 46 | | 44 | |
| Drops 30 | 45 | 300 | 42 | 280 |
| Drops 40 | 38 | 220 | 32 | 200 |
| Drops 50 | 28 | 180 | 28 | 160 |
| Drops 60 | 28 | 170 | 26 | 150 |
| Drops 70 | 26 | 150 | 26 | 140 |
| Drops 80 | 24 | 140 | 24 | 130 |
| Drops 90 | 23 | 125 | 24 | 120 |
| Drops 100 | 22 | 120 | 22 | 120 |

As shown in the comparison of Tables 1 and 2, testing was performed to determine the difference in electrical resistance between distilled water, a basic liposome dissolved in distilled water, and the SK713 SLP Prodosomes dissolved in distilled water, and the trace mineral concentrate in pure form that is used in the processing of the prodosomes. A standard multi-meter (Armaco Brand 20A) was used and was set to measure ohms with a digital output. Ohms are a measurement of the electrical resistance that can be found in a particular solution or compound. Tests were run on both the 100× and 1000× setting, the 1000× setting being more sensitive to ionization. Pure distilled water was used as a control, and as the medium for dissolving the various liquids to be tested. All materials including the distilled water were allowed to reach room temperature. The amount of water used in each test was a volume of 50 mL and all came from a single bottle. All containers used for testing were glass. In all cases, each material to be tested was added 1 drop at a time into the water and the multimeter was used to detect resistance as determined by Ohm readings. After the initial tests were completed, identical testing was repeated to ensure uniformity of results.

In measuring pure distilled water, the detection of ohms, as shown on the digital readout, at the 100× setting, was not detectable, indicating infinite resistance and therefore no conductivity. At the 1000× setting, the reading was 600 (Table 1).

Next, the basic liposome was added 1 drop at a time to 50 mL of distilled water, with an Ohm reading being taken after each drop was manually stirred in the water (Table 1). On the 100× setting, there was no evidence through the multimeter readings to show any Ohms, and therefore no electrical conductivity, even up to 100 drops of the liposome solution in the water medium, confirming the electroneutrality of the phospholipid molecules. At the 1000× setting, 1 drop lowered the resistance from the 600 level to 400, 2 drops only changed the reading to 380, the same with 3 drops, while 4 drops of liposome lowered only to 360, and 5 drops to 350. At 6-9 drops the reading was maintained at 300 Ohms (Table 1).

Next, the SK713 SLP Prodosome was added 1 drop at a time to 50 mL of distilled water, with an Ohm reading being taken after each drop was manually stirred in the water (Table 2). At the 1000x setting, 1 drop lowered the resistance from the 600 level to 350, 2 drops changed the reading to 220, the same with 3 drops, lowering the resistance level to 220. Four drops of Prodosome decreased the reading to 140, and stayed the same at 5 drops. At 6-8 drops, the reading was maintained at 120 while the 9th drop of Prodosome lowered the Ohms to 100. After the second drop of Prodosome was added to the water medium and tested, and as subsequent tests were performed, the decrease in resistance and concurrent increase in conductivity over the basic liposome was approximately 3 times as great. Additionally, in comparing the Ohms reading of the basic liposome to the Prodosome testing after each drop 10-30, the increase in conductivity of the Prodosome material was consistently 3-4 times more than the basic liposome. Also, with the basic liposome being added up to 100 drops in the water, there was no evidence of lessening resistance and therefore no conductivity at the 100x setting. On the other hand, the Prodosome test done with the 100× setting on the multimeter did begin to show a lessening of resistance according to Ohms at drop number 30 and continued to gradually decrease in resistance at testing of drops 30-100.

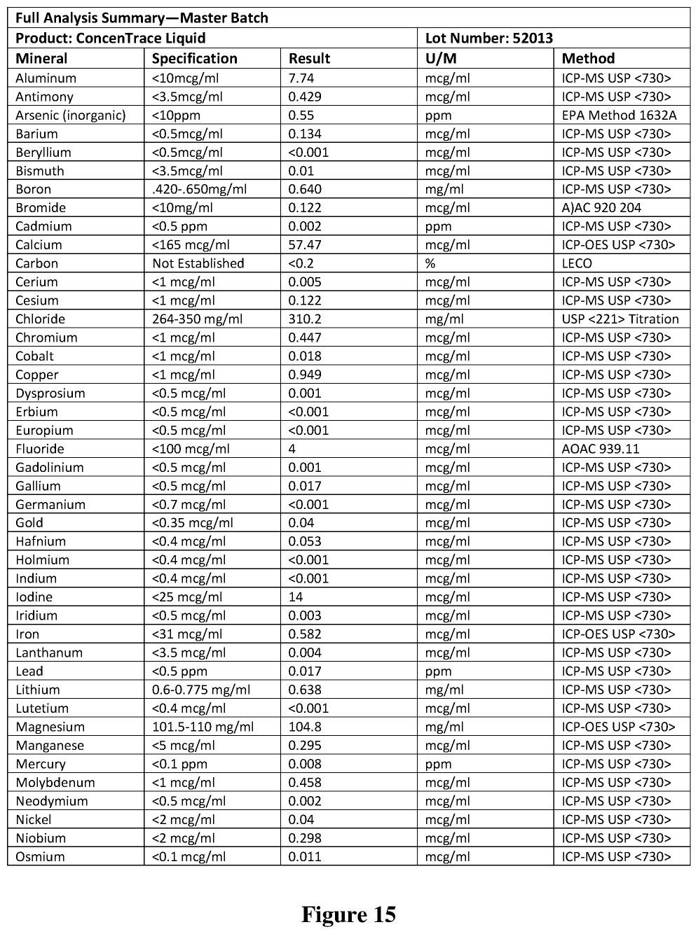

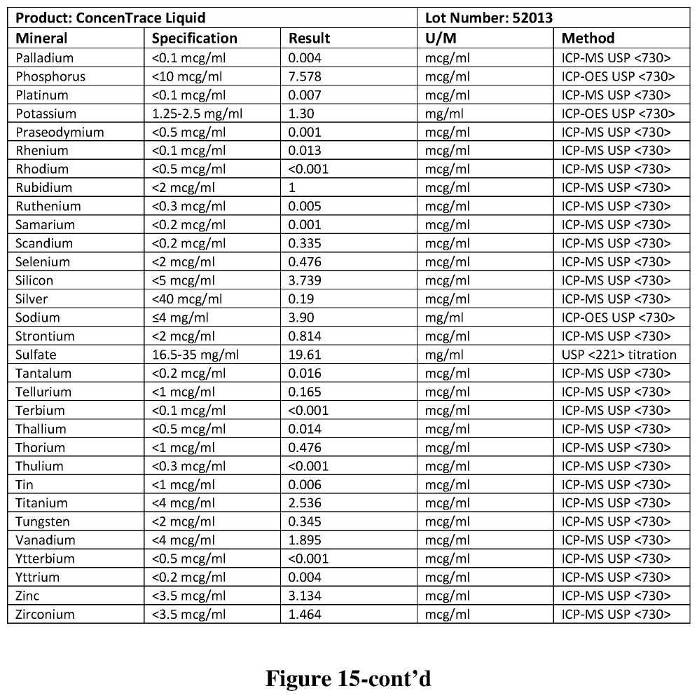

Finally, the pure trace mineral concentrate (“TMC”) used in the production of the Prodosome was added to the distilled water in an amount equivalent to that found in the same volume of Prodosome (Table 3). In other words, the TMS was added to a pre-measured quantity of water at a fraction of the total volume of Prodosome so as to ensure the amount of TMC would be the same as exists in the Prodosomes at each measurement, drop for drop, comparing the Prodosome to the TMC. In this test, the ionization was strong enough to only require the multimeter to be used at the 100× setting. At 1 drop of the TMC, the reading was 1 drop 500, 2 drops 280, 3 drops 300, 4 drops 160, 5 drops 140, 6 drops 120, and drops 7 and 8 at 100. The minerals contained in the TMC are listed in FIG. 15.

TABLE 3
Electrical Resistance of Solutions Containing
Pure Ionic Trace Minerals

| Sample #1 Pure Ionic Trace Minerals | 100X Setting (in Ohms) |
|---|---|
| Drops 1 | 500 |
| Drops 2 | 280 |
| Drops 3 | 200 |
| Drops 4 | 160 |
| Drops 5 | 140 |
| Drops 6 | 120 |
| Drops 7 | 100 |
| Drops 8 | 100 |
| Drops 9 | 80 |
| Drops 10 | 80 |
| Drops 11 | 75 |
| Drops 12 | 75 |
| Drops 13 | 65 |
| Drops 14 | 65 |
| Drops 15 | 65 |
| Drops 16 | 65 |
| Drops 17 | 65 |
| Drops 18 | 65 |
| Drops 19 | 65 |
| Drops 20 | 60 |
| Drops 21 | 45 |
| Drops 22 | 40 |
| Drops 23 | 38 |

The readings of the multimeter in Ohms for the Prodosomes versus the TMC were consistently less by an order of magnitude (10×), drop for drop. Again, while there was an order of magnitude greater drop in resistance from the TMC, the concentration of ions in both the TMC/water mixture and the Prodosomes/water mixture was the same. This suggests strongly that the Prodosome material is acting as an effective insulator (i.e., “biological capacitor”), and is evidence of the electrical activity showing in the Prodosome material only coming from the ionic minerals contained on the outermost phospholipid layer of the Prodosome clustoidal sphere. Being neutral, the distilled water medium does not allow the Prodosome sphere to completely disintegrate, therefore the balance of the ionic material would be contained, or insulated, in the lower levels of the multi-lamellar clustoidal spheres. This would also promote the benefits of the conductivity supplied by the release of the infused ionic TMC to be sustained over an extended period of time, as each layer of the multi-lamellar clustoidal Prodosome sphere sequentially disintegrates in the more alkaline environments of the body (i.e., mouth, intestine, and possibly blood).

The biological capacitor function of the multilamellar SK713 SLP clustoids would not occur in normal liposomes (See Table 1) and is only possible because of the energy frequency imprinting (also known as “EFlquence” technology) of the SK713 SLP process technology. The novel multilamellar clustoidal phospholipid encapsulation of embodiments of the present invention (SK713 SLP/Prodosomes) was developed to facilitate more stable, competent, and comprehensive synchronized absorption, and synchronized bioavailability and bioactivity of orally ingested nutrition. The SK713 SLP is distinctly and surprisingly unique and superior to any previous liposomal technologies and, unlike previous versions: contains more phospholipid substrate, which is impregnated and saturated with solar dried electrolytes in an ionic state; is demonstrably and significantly more stable; and is consistently more uniform and shown to be more efficacious than other liposomal technologies tested.

Clustoidal Multilamellar SLP Encapsulated Nutraceutical Multivitamin Formulations (SK713 SLP Encapsulated VMP35 Multinutrient Complex), i.e., Prodovite®.

In one embodiment of the present invention, the prodosomes based multivitamin formulation induced a beneficial effect on the properties of human blood by promoting rapid delivery of their nutritional contents to a human subject in vivo. In a particular embodiment, a novel clustoidal multilamellar soy-lecithin-phospholipid encapsulation formulation (“SK713 SLP Encapsulated VMP35 Multinutrient Complex” or “VMP35 MNC”), which includes, among other ingredients, vitamins, such as vitamins A, C, D3, E, B1, B2, B3, B6, and B12. The formulation was designed to be administered transmucosally. The components of VMP35 MNC Formulation are described in Table 4. However, the transmucosal route of administration of this formulation was not intended to be limiting. As understood by a person skilled in the art, the studied multivitamin formulation is also suitable for other routes of oral administration. Testing results showed that VMP35 MNC is a superior nutraceutical supplement that is able to effect positive changes in morphological, hematological, and rheological properties, and to overcome the limitations of those with various underlying digestive inefficiencies. See, e.g., Y. Shoji, et al., Nutraceutics and delivery systems, 12 J. DRUG TARGETING 385 (2004), incorporated by reference herein in its entirety.

TABLE 4
SK713 SLP Encapsulated VMP35 Multivitamin,
Mineral & Phytonutrient Formulation

| | Per | Unit of |
|---|---|---|
| INGREDIENT | Serving | Measure |
| R/O water | 26300 | mg |
| Vitamin A (Retinyl Palmitate) | 5000 | IU |
| Vitamin C (Ascorbic acid) | 60 | mg |
| Vitamin D3 (Cholecalciferol) | 0.025 | mg |
| Vitamin E (Alpha-tocopheryl | 15 | IU |
| Succinate) | | |
| Vitamin B1 (Thiamin HCl) | 1.5 | mg |
| Vitamin B2 (Riboflavin) | 1.7 | mg |
| Vitamin B3 (Niacin) | 20 | mg |
| Vitamin B6 (Pyridoxine HCl) | 2 | mg |
| Folic acid/Folate | 400 | mcg |
| Vitamin B12 (Cyanocobalamin) | 50 | mcg |
| Biotin | 300 | mcg |
| Pantothenic acid (d-calcium | 10 | mg |
| pantothenate) | | |
| Calcium lactate | 100 | mg |
| Iodine (potassium iodide) | 0.15 | mg |
| Magnesium citrate | 100 | mg |
| Zinc sulfate | 10 | mg |
| Sodium selenite | 0.07 | mg |
| Copper gluconate | 1 | mg |
| Manganese sulfate | 2 | mg |
| Chromium chloride | 0.12 | mg |
| Potassium citrate | 99 | mg |
| Choline bitartrate | 20 | mg |
| Inositol | 20 | mg |
| White pine cone extract | 5 | mg |
| BiAloe Concentrated 200:1 | 20 | mg |
| Water Extract | | |
| VMP35 1:1 Water Extracted | 1700 | mg |
| Herbal Blend: | | |
| Astragalus Root extract | 1:1-247.5 | mg |
| Ginger Root extract | 1:1-99.95 | mg |
| Green tea Leaf extract | 1:1-199.92 | mg |
| Fo ti Root extract | 1:1-199.92 | mg |
| Hawthorne berry extract | 1:1-150.96 | mg |
| Elderberry extract | 1:1-99.95 | mg |
| Eleuthero Root extract | 1:1-150.96 | mg |
| Chamomile Flower extract | 1:1-199.92 | mg |
| Citrus bioflavonoids (from | 1:1-199.92 | mg |
| rose hips) | | |
| Gotu kola Leaf extract | 1:1-150.96 | mg |
| SK713 SLP | 342 | mg |

One of the major components of VMP35 MNC formulation is a specially prepared high grade soy lecithin material that contains a minimum of 85% phosphatidylcholine (“>85PC”), an essential phospholipid, while most lecithin products contain only 19-21% PC. See, e.g., C. R. Scholfield, Composition of soybean lecithin, 58 J. AM. OIL CHEMISTS' SOC'Y 889 (1981), incorporated by reference herein in its entirety. The high PC content in SK713 SLP ensures thorough formation of liposomes. In addition to acting as biological capacitors and protecting the nutritional contents, multilamellar liposome phospholipids offer several health-related benefits. Due to their role in molecular transport, phospholipids also influence cell growth and development, and speed up organism regeneration after physical exertion. Phospholipids limit cholesterol absorption from the gastrointestinal tract and are beneficial in liver therapy, for instance, in the treatment of steatosis. Phospholipids inhibit inflammation factors, some of which are pathogens of the alimentary canal and promoters of cancers, for example adenoma, and colon cancer. See, e.g., A. Ambroziak, et al., Milk phospholipids as nutraceutic, 34 POLSKI MERKURIUSZ LEKARSKI 62 (2013), incorporated by reference herein in its entirety.

The multi-lamellar or multisphered-multilayered-clustoidal structure of SK713 SLP, unlike standard liposome technology, is capable of encapsulating a diverse range of nutrients simultaneously. Through experimentation, SK713 SLP was found to form vesicles made up of hundreds of concentric lipid bilayers that range in size from 100 nanometers to 500 micrometers and are made up of a few dozen to several thousand molecules. See, e.g., B. C. Keller, Liposomes in nutrition, 12 TRENDS IN FOOD SCI. & TECH. 25 (2001), incorporated by reference herein in its entirety. As soon as the concentration of phospholipids reaches critical mass, the water-repelling ends organize to form the liposomes with the lipophilic (fat-attracting) hydrocarbon chains oriented inwards and the hydrophilic (water-attracting) groups facing outwards, forming the lipid bilayer structure.

The SK713 SLP multilamellar liposomes form spontaneously as the electrostatic and adsorptive properties lower surface tension (surfactant). The net result is thorough and complete phospholipid encapsulation (or entrapment) of nutritional ingredients within multiple layers of nano- to low micrometer-sized spheres. This electrostatic encapsulation is effective for encapsulating and transporting both water and fat-soluble nutritional ingredients including phytonutrients within the same spherical structure. See, e.g., A. Akbarzadeh, et al., Classification, preparation, and applications, 8 NANOSCALE RESEARCH LETTERS 102 (2013); W. Helfrich, Size distributions of vesicles: the role of the effective rigidity of membranes, 47 J. PHYSIQUE 321 (1986); incorporated by reference herein in its entirety.

TABLE 5
Phytonutrients in VMP35

| Standardized | Chemical | Physiological Performance |
|---|---|---|
| Botanicals | Constituents | and Metabolic Function |
| Astragalus | Flavonoids, | An adaptogen |
| membranaceus | saponins, | An antioxidant and immunomodulator |
| (family | phytosaccharides, | Regulate metabolic homeostasis and energy |
| Fabaceae) | amino acids and | metabolism |
| | isoflavan | Potent enhancement of immunity and |
| | glycosides | immunostimulatory properties |
| | | Regeneration and revitalization of tissue |
| Polygonum | Flavonoids, | Antioxidant, anti-ageing, inhibits anaerobic |
| multiflorum | stilbenes, alkaloids, | events and organisms, helps protect DNA |
| (family | and quinones | and prevent cell mutations, supports proper |
| Asparagaceae) | | blood lipid levels and protects the structure |
| Known as Fo-Ti | | of cells in the endothelium, reducing |
| Root | | scarring. |
| | | Enhance brain function |
| | | Rejuvenate nerve, brain cells and endocrine |
| | | and adrenal glands |
| | | Fortify muscles |
| | | Enhance detoxification |
| Camellia | Structurally diverse | Antioxidant, reduces the need for inducing |
| sinensis (family | polyphenols and | inflammatory events, anti-photoaging and |
| Theaceae) | catechins especially | autophagy |
| Known as Green | (−)-Epigallocatechin | Neuroprotectant |
| Tea | gallate (EGCG) | Enhances brain function, cognition and |
| | | mood alleviator |
| | | Stress resistance |
| | | Reduces tissue fragility and promotes |
| | | connective tissue repair |
| Matricaria | Flavonoids, | Improves muscle tone |
| chamomilla | sesquiterpenes, | Reduces the potential for spasms |
| (family | coumarins, and | Improves connective tissue strength |
| Asteraceae) | polyacetylenes | Reduces the need for inducing inflammatory |
| Known as | including herniarin, | events |
| Chamomile | umbelliferone, | Calming and stress relieving |
| Flower | chlorogenic acid, | Promotes restful restorative sleep |
| | apigenin, luteolin, | |
| | quercetin, rutin, | |
| | and naringenin | |
| Rosa canina | Citrus | Antioxidant |
| (family | bioflavonoids, | Reduces the need for inducing inflammatory |
| Rosaceae) | triterpenoids, and | events |
| Known as Rose | phytosterols | Promotes joint health & function |
| hips | | Promotes tissue strength and integrity, |
| | | increasing tissue resistance to allergenic |
| | | insults |
| | | Promotes connective tissue repair |
| | | Repair, re-structure, regenerate, re-vitalize |
| | | and re-build tissues |
| Eleutherococcus | Flavonoids, | An adaptogen |
| senticosus | glycosides | Strengthens immune function |
| (family | | Enhances endurance |
| Araliaceae) | | Boosts stamina and energy |
| Known as | | Strengthens bones |
| Eleuthero root | | Reduces fatigue |
| Crataegus | Flavonoids | Promotes cardiovascular functions |
| oxyacantha | (vitexin, rutin, | Supports digestion |
| (family | quercetin, and | Reduces fatigue |
| Rosaceae) | hyperoside, | Promotes improvements in exercise |
| Known as | flavone-C, | performance, tolerance, and breathing |
| Hawthorn berry | epicatechin), | |
| | oligomeric | |
| | proanthocyanidins | |
| | (procyanidin B-2), | |
| | triterpene acids | |
| | (ursolic-, oleanolic-, | |
| | crataegolic acids), | |
| | and phenolic acids | |
| | (caffeic acid), | |
| | chi orogenic acid | |
| Centella asiatica | Triterpenoides, | Antioxidant, reducing the need for inducing |
| (family | glycosides | inflammatory events |
| Apiaceae) | (asiaticosides, | Cardioprotectant |
| Known as Gotu | asiatic acid, | Boosts immune functions |
| kola leaf | madecassic acid, | Boosts metabolic homeostasis |
| | madecassoside, | Promotes healthy aerobic metabolism |
| | centellin, asiaticin | Supports proper blood lipid levels, and |
| | and centellicin), | protects the structure of cells in the |
| | polyacetylenes, | endothelium, reducing scarring. |
| | vitamin K, | Promotes proper blood sugar utilization, |
| | magnesium, | Promotes the health of cardiovascular |
| | calcium, sodium | tissues, improves cellular energy output. |
| | and potassium | Promotes tranquility |
| | | Relieves stress |
| | | Promotes improved intelligence |
| | | Improves mental functions focus, |
| | | concentration, and memory |
| | | Strengthen adrenals |
| | | Exerts calming effect and support restful |
| | | sleep |
| Zingiber | Phenolic acids and | Antioxidant; reduces the need for inducing |
| officinale (family | terpenoids, | inflammatory events |
| Zingiberaceae) | gingerols, shogaols, | Boosts immune functions and blood |
| Known as Ginger | and paradols | circulation |
| Root | | Promotes digestive health |
| | | Boosts muscle performance, muscle strength, |
| | | and overall sports performance |
| | | Reduces muscle damage and delayed onset |
| | | to muscle soreness |
| Sambucus nigra | Bioflavonoids | Antioxidant and reduces the need for inducing |
| (family | | inflammatory events. |
| Adoxaceae) | | Promotes improved tissue structural strength |
| Known as | | and functional competence, protecting against |
| Elderberry | | viral insult, allergic vulnerability and damage |
| | | from trauma |
| | | Supports respiratory health |
| Standardized | Acemannan | Immunomodulatory activities |
| DSR0114 Aloe | [acetylated | |
| barbardensis | mannans, | |
| (Inner Leaf water | monoacetyl | |
| extracted freeze- | mannose polymers | |
| dried gel | with β-(1,4)-D- | |
| powder) | linkage] | |
| [Organic | | |
| BiAloe ® | | |
| DSR0114] | | |
| Known as Aloe | | |
| vera | | |
| Standardized | Poly- | Promotes improved tissue structural strength |
| LPC108 Pinus | phenylpropanoid | and functional competence, protecting against |
| sylvestris | polysaccharide | viral insult |
| Proligna ® Freeze | complex | Helps diminish the extent of damage from |
| Dried LPC108 | | trauma |
| Scotch Pine | | Restore aerobic cellular metabolism creating |
| Cone freeze | | an adverse environment for anaerobes, i.e. |
| dried powder | | yeasts, parasites, etc. |
| Known as White | | Promote an aerobic cellular environment to |
| pine cone | | protect DNA and promote normal cell |
| | | structure and function, preventing cell |
| | | mutations. |

TABLE 6
Vitamins in VMP35

| | | Physiological Performance | |
|---|---|---|---|
| Vitamins | Constituents | and Metabolic Function | |
| Vitamin B Complex, a | Composed of eight B- | 1. | Cellular health and |
| combination of eight water | vitamins including vitamin B1 | | integrity |
| soluble vitamins | (thiamine), | 2. | Growth of red blood cells |
| | B2 (riboflavin), B3 (niacin), | 3. | Boosts energy level |
| | B5 (pantothenic acid), B6 | 4. | Promotes cardiovascular |
| | (pyridoxine), | | health |
| | B7 (biotin), B9 (folic acid), | 5. | Boosts ocular health and |
| | and B12 (cyanocobalamin) | | neuronal functions |
| | Note: Methylcobalamin is | 6. | Boosts digestion and |
| | unstable in a liquid (rapidly | | healthy appetite |
| | deconjugating) and therefore | 7. | Optimizes hormones and |
| | inappropriate for use in the | | cholesterol synthesis |
| | VMP35. | 8. | Strengthens muscular |
| | | | integrity and muscle tone |
| | | 9. | Fetal brain development |
| | | | and reduces of birth defects |
| Vitamin A, a fat soluble | Retinol, Retinyl esters and | 1. | Promotes ocular and bone |
| vitamin | provitamin A carotenoids | | health |
| | | 2. | Boosts immune |
| | | | competence |
| | | 3. | Protects against night |
| | | | blindness and age-induced |
| | | | decline |
| | | 4. | Promotes healthy growth |
| | | | and reproductive health |
| | | 5. | Reduces the risk of acne |
| Vitamin C, a water soluble | Ascorbic acid; Ascorbates | 1. | A potent antioxidant |
| vitamin | | 2. | Boosts immune competence |
| | | 3. | Promotes ocular and |
| | | | cardiovascular health |
| | | 4. | Maintains healthy blood |
| | | | pressure |
| | | 5. | Ameliorate iron deficiency |
| Vitamin D | Also, known as Calciferol, | 1. | Maintains healthy bones |
| (Classified as a prohormone, | Cholecalciferol, and 1,25- | | and teeth. |
| or precursor of a hormone) | Dihydroxycholecalciferol. | 2. | Supports Immune and |
| | | | Neuronal Health. |
| | | 3 | Boosts Pulmonary and |
| | | | Cardiovascular Functions |
| | | 4. | Insulin Sensitizer |
| | | 5. | Modulates genes involved |
| | | | in cancer development |
| Vitamin E | Alpha-tocopherol | 1. | Promotes healthy skin and eye |
| | | 2. | Boosts cardiovascular health |
| | | 3. | Promotes insulin sensitivity |

TABLE 7
Macronutrients and Micronutrients in VMP35

| Micronutrients and | | Physiological Performance | |
|---|---|---|---|
| Macronutrients | Constituents | and Metabolic Function | |
| Calcium | Calcium lactate | 1. | Strong bones and teeth |
| | | 2. | Mobilizes skeletal muscle |
| | | 3. | Stabilizes blood pressure |
| | | 4. | Acts as a pH buffer in the ion pool |
| Iodine | Potassium iodide | 1. | Promotes disinfections in brain and other |
| | | | tissues |
| | | 2. | Promotes cardiovascular, immune, and |
| | | | thyroid health |
| | | 3. | Boosts metabolism |
| | | 4. | Neuronal development during pregnancy |
| Selenium | Sodium selenite | 1. | Scavenges oxygen free radicals |
| | | 2. | Reduces DNA damage |
| | | 3. | Prevents cellular injury |
| | | 4. | Boosts immune competence |
| | | 5. | Promotes cardiovascular health |
| | | 6. | Reduces inflammatory response |
| | | 7. | Protects against neurological injuries |
| Copper | Copper gluconate | 1. | Promotes cellular respiration and antioxidant |
| | | | defense |
| | | 2. | Enhances production of red blood cells |
| | | 3. | Maintains neuronal health and |
| | | | neurotransmitter functions |
| | | 4. | Boosts immune health |
| | | 5. | Helps synthesize collagen |
| | | 6. | Promotes energy homeostasis |
| | | 7. | Builds and repairs connective tissues |
| Magnesium | Magnesium lactate | 1. | Boosts muscle performance including |
| | | | muscle contraction, functioning and |
| | | | relaxation |
| | | 2. | Promotes bone and dental health |
| | | 3. | Enhances neuronal functions |
| Chromium (III), an | Chromium chloride | 1. | Boosts glucose and lipid metabolism |
| essential trace | | 2. | Enhances insulin sensitivity |
| element | | 3. | Promotes lean body mass |
| | | 4. | Boosts metabolism |
| | | 5. | Essential for lipid, fat and carbohydrate |
| | | | metabolism |
| | | 6. | Lowers blood cholesterol |
| Potassium | Potassium citrate | 1. | Boosts energy level and diverse |
| | | | physiological functions |
| | | 2. | Metabolizes sugar |
| | | 3. | Enhances energy production |
| | | 4. | Potentiates muscular integrity and functions |
| | | 5. | Promotes cardiovascular health |
| Zinc | Zinc Sulfate | 1. | Maintains structural integrity |
| | | 2. | Acts as a membrane stabilizer |
| | | 3. | Enhances cellular growth, metabolism, and |
| | | | replication |
| | | 4. | Boosts immune competence |
| | | 5. | Boost sexual competence and reproductive |
| | | | health |
| | | 6. | Essential for neurological well-being and |
| | | | integrity |
| | | 7. | Promotes wound healing |

a. Encapsulation of Nutrients

One of the limitations of encapsulating nutrients within the SLP transport spheres is the relative insolubility of some ingredients in water. Many nutritional compounds, especially inorganic minerals and resinous phytonutrients, are not readily soluble in water. To overcome this obstacle, prior to SK713 SLP processing, all materials are pre-processed in a low sheer tri-blender using jet-compression-particle-processing technology. This step is akin to a wet-milling process. In essence, the nutritional/nutraceutical materials are added directly to distilled water. The admixture is then blended at a low and consistent speed for a specific time, depending on the viscosity of the liquid and the physical and chemical properties of the added components. At the same time, water is circulated to create a secondary motion. No excess heat is produced in the mixing process. The low heat production combined with low shear used in the mixing step preserves the physicochemical stability of the nutrients and botanicals contained within the solution or suspension. The process continues for a period of time to substantially reduce particle size and to achieve consistency and uniformity of the mixed materials over successive batches. The electrolyte-impregnated SK713 SLP compound is then added to encapsulate these nutraceutical particles with greatly reduced particle size. Importantly, this preparation greatly improves bioavailability of the nutrients and botanicals. This preparation further ensures that previously insoluble materials can now be blended and dispersed into a semisolid or even a liquid state. The liquid concentrate is made up of the high-grade lecithin (>85% PC) combined with an amount of alcohol in exact proportions and blended at specific speeds for a specified time to achieve a solution with the right consistency, viscosity, and grade of material. The SK713 SLP material can then be blended into the liquid nutritional compound under precisely required speeds and blending times based on the material in the supplement as well as the batch size. The same process can be utilized for preparing topical formulation to achieve enhanced delivery. The amphipathic (hydrophilic and hydrophobic) properties of SK713 SLP allow it to encapsulate nutraceutical ingredients contained in a liquid medium and to serve as an efficient transmembrane delivery vehicle for these nutrients. The SK713 SLP delivery vehicles or spheres as set forth above comprise all natural generally recognized as safe (“GRAS”) ingredients or pharmaceutically/nutraceutically acceptable ingredients, which are suitable for human consumption.

b. Multilamellar Sphere Components

The SK713 SLP multilamellar spheres contain large quantities of electrolytes and hydroxyl-rich botanicals that contribute bioflavonoids and assist in maintaining healthy pH, proper hydration, and the transport and utilization of vital nutrients. The SK713 SL phospholipid spheres are zwitterions, methyl donors, and potential alkalinizing buffers. See, e.g., G. Bouchard, et al., Theoretical and experimental exploration of the lipophilicity of zwitterionic drugs in the 1,2-dichloroethane/water system, 19 PHARM. RESEARCH 1150 (2002), incorporated by reference herein in its entirety. Zwitterions carry both positive and negative charges and may lower the energy requirement for transporting molecules thereby enhancing absorption by spreading the nutrient out over a larger surface area.

Zwitterions are soluble in many solvents, e.g., water. The SK713 SL phospholipid spheres have a natural “adhesive” property that enhances the ability of the body to absorb their nutritional contents. Specifically, in certain embodiments, the present invention relates to a novel soy-lecithin-phospholipid-nutrient encapsulation technology, which could achieve rapid onset and improved bioavailability of the nutrients encapsulated within clustoidal multilamellar soy lecithin (SK713 SLP) structures.

c. Live Blood Cell Imaging

Live blood cell imaging was performed using an Olympus BX-30 light microscope with a Phase Contrast Condenser to visualize samples. A 150 watt lightbox with fiber optic cable assembly was used to highlight the specimen against a gray field and increase the range of intermediate shades. The lighting produces a high level of cell definition, clearer morphology, and can distinguish features of some cell walls. The lens configuration was 10× eyepiece and 100×-oil-immersion objective magnification to achieve approximately 1000 times magnification. Oil immersion achieved finer resolution and brightness.

d. Peripheral Blood Smear Test

Peripheral blood smear was performed by puncturing the finger with a Bayer Single-Let Disposable Lancet 23G 2.25 mm sterile single-use lancing device. A small amount of capillary blood was allowed to exude and collect spontaneously on the fingertip without squeezing the finger. The blood was transferred directly onto a microscope slide without touching the slide with the finger. The slides used were pre-cleaned standard 1-inch by 3-inch with a thickness of 1 millimeter supplied by Electron Microscopy Sciences. The slide was covered quickly and gently with a cover glass without pressure to protect blood cells from damage. The cover glass was pre-cleaned #1 22 mm×40 mm with 0.13- to 0.17-mm thickness, supplied by Electron Microscopy Sciences. The corners of the cover glass were tapped carefully to disperse surface tension and create an even layer for viewing. The slide was then transferred directly to the microscope for viewing. Evaluation of blood properties began in less than 30 seconds after the blood was taken from the finger. Consistent blood extraction and handling procedures were followed to avoid artifacts.

This test is not intended for any diagnostic evaluations as this imaging technology has not been considered appropriate for such applications. Much controversy has arisen over the use of PBS LBCI due to non-adjudicated commercial use, unsubstantiated extrapolations, over-reach, and ambiguity of interpretative criteria for diagnostic purposes. The objective of using PBS LBCI in embodiments of the present invention was, however, to serve as a time-sensitive marker of biological perturbation and as a visual analytical tool only for the degree of responsiveness of human blood to the delivered bioactive nutrients. As such, the central finding is not the nature of the changes themselves per se, but the extent to which the changes occurred in contrast to the control and baseline groups.

e. Effects on Human Blood

The SK713 SLP encapsulated VMP35 multivitamin formulation was administered transmucosally to thirty-eight (38) human subjects, both males and females, ranging in age from 12 years to 82 years. The blood samples drawn from the testing subjects administered with VMP35 MNC formulation were analyzed and compared to those drawn from subjects in control group, who were administered commercially available bottled water. The evaluation demonstrates that the SK713 SLP delivery exerts rapid positive effects on morphological, hematological, and rheological properties of the blood. The rapid onset of transmucosally administered VMP35 MNC formulation also suggests that the SK713 SLP technology efficiently delivers nutrients into the blood via the sublingual mucosa and may overcome digestive inefficiencies in vivo. See, e.g., A. Akbarzadeh, et al., Classification, preparation, and applications, 8 NANOSCALE RESEARCH LETTERS 102 (2013); A. Akbarzadeh, et al., Synthesis, characterization and in vitro studies of doxorubicin-loaded magnetic nanoparticles grated to smart copolymers on a 549 lung cancer cell line, 10 J. NANOBIOTECHNOLOGY 46 (2012); A. Valizadeh, et al., Quantum dots: Synthesis, bioapplications, and toxicity, 28 NANOSCALE RESEARCH LETTERS 480 (2012); each of which is incorporated by reference herein in its entirety.

As set forth above, the presence of embedded free ions in SK713 (prodosome) enhances bio-electrical properties of the liposomal delivery system in an aqueous solution (See Table 2) and in the blood making it superior to conventional phospholipids in terms of its conductive properties and biological compatibility and functionality. Without being bound by any theory, it is expected that the loading of ions and other nutritional ingredients greatly increases the absorption of nutrients and promotes synergistic effectiveness of the simultaneously absorbed nutrients. The molecular structure created in the SK713 liposomal delivery system acts like a biological capacitor that can transport a variety of nutrients simultaneously across the sublingual mucosal membranes in the mouth and/or the wall of the small intestine into the portal circulation.

Application of Prodosome (SK713 SLP) Delivery System in Oral Administration

It is likely that the SK713 SLP spheres provide protection of the encapsulated nutritional contents within the multilamellar structures against the harsh acidic environment in the stomach. This protection enables the nutrients within the spheres to reach the small intestine intact, which promotes greater nutritional synergy in absorption and utilization. The entire SK713 SLP process helps to create a formulation that enables nutrients to disperse over a larger surface area within the small intestine. Initially, the low-sheer tri-blender jet compression technology decreases particle size of larger and more granular or resinous materials. The smaller particle size of a particular nutrient will allow this nutrient to cover a broader surface area once it reaches the small intestine. In addition, encapsulation within the SK713 SLP spheres can decrease particle size even further, especially of fat-soluble vitamins and phytonutrients. As the remaining mass of nutrients that does not absorb through the sublingual mucosa reaches the small intestine, it is likely to be absorbed through diffusion across the epithelial wall of the small intestine. The process of decreasing particulate size of these nutrients allows the entire mass of nutrients to disperse over a larger area of the small intestinal wall. This dispersion greatly increases the surface area into which nutrients can be absorbed so that less of the nutritional intake passes into the large intestine for elimination.

The multi-lamellar prodosome compositions and methods described above, the effect of prodosome-encapsulated VMP35 MNC on human blood may be further understood in connection with the following Examples. In addition, the following non-limiting examples are provided to illustrate the invention.

### EXAMPLE 1

Method of Producing Clustoidal Multilamellar Soy Lecithin Phospholipid (SLP)

Step 1. Generally, a nutritional, nutraceutical, or pharmaceutical active ingredient substrate is processed through an advanced wet milling/particle compression process to facilitate a type of mechanical predigestion of substrate that enables more of the substrate to be encapsulated in the phospholipid spheres. Thoroughly wet milling the substrate significantly increases surface area of the substrate and enables a higher concentration and wider range of substrate ingredients to be homogenized and encapsulated in the Prodosome process.

The following steps are done in relatively small batches (approximately 5-gallon containers) to achieve an optimal speed ensuring the most complete and thorough homogenization of constituents. Following each step below, blending should be performed in small circular motions in the opposite direction of the rotation (counter-rotation) of the blender blade to increase the torsion to effect the interaction of ions with phospholipids over a greater fluid surface area and produce an energetically enhanced homogeneous mixture. Generally, start with an amount of water between 40-80% of total final volume. Heat water to a temperature between 90 degrees F. and 140 degrees F.

Step 2. In a 5-gallon stainless steel drum of water, solar evaporated mineral/trace mineral liquid concentrate between 1 to 120 g/kg of water was mixed in at a level ranging from 0.1% to 12.0%. this mixture was blended for a time between 1-5 minutes at a speed between 3,000 and 25,000 RPM in a high-RPM spinning vortex of water between 300 and 800 g/kg of total mixture to completely and uniformly disperse ions into what is now “structured water.” (Trace mineral liquid concentrate is available from Trace Minerals Research, Ogden, Utah, United States; see also FIG. 15).

Step 3. High-grade lecithin containing >85% Phosphatidylcholine (“PC”) at between 2 and 200 g/kg of the total mixture was added, corresponding to between 2% to 20%, respectively, and thoroughly mixed into the ion-rich water, blended between 1 and 5 minutes at a speed from 3,000 to 25,000 RPM, depending on substrate viscosity. Then, a small amount of ethyl alcohol was added (not less than 150 proof), at between 50 and 450 g/kg of the total mixture, and blending continued between 1 and 5 minutes at a speed of between 3,000 and 25,000 RPM depending on substrate viscosity. The mixture is then allowed to cool. As a result, the phospholipid structures are completely impregnated and saturated with free ions, achieving a completely homogeneous mixture of electrolytically “charged” SK713 SLP material.

Variants of the procedure include: adding between 2 and 20% amounts of phosphatidyl choline with a PC content of no less than 70%. Adding between 5 and 45% USP Alcohol, at a level no less than 150 proof. The mixing procedure can include ultrasonic mixing.

Step 4. This mixture is then added to the nutritional, nutraceutical, or pharmaceutical active ingredient substrate of Step 1 in a blender and blended thoroughly to facilitate complete encapsulation of the substrate. In embodiments of the present invention, a level of between 0.5% and 10% can be used in “prodosoming” finished products depending on the composition and state (aqueous or dry) of the substrate being encapsulated.

The process may be varied slightly, within a narrow parameter, as to the degree of phosphatidyl choline (PC) content, depending on the end usage required. Limited variance of PC content of finished Prodosome may alter viscosity of liposomal material without creating any loss of advantage. Differing viscosity prodosomes may be required depending upon active ingredient intended for encapsulation, such as material more or less soluble, or materials containing higher levels of lipids. Trace mineral concentrate amounts can also be varied to some extent, depending on the substrate and benefit endpoints.

This mixing process evidently catalyzes association between electrolytes and other molecules within the total substrate (i.e., methyl and phosphoryl groups); certain B vitamins with methyl and/or phosphoryl ligands; also facilitating the permeation of substrate material into the phospholipid intermolecular spaces of the Prodosomes.

This process enables comprehensive and uniform encapsulation of nutritional and/or pharmaceutical ingredients in the SK713 SLP phospholipid prodosome capsules, facilitating superior absorption of nutritionally and pharmacologically active therapeutic substances that provide benefits following absorption of the energetically enhanced electrolyte-impregnated phospholipids.

The present disclosure includes specific materials with exacting levels of each, blended with distinct sequence and timing. The SK713 sphere is unique in many aspects, as follows.

A. Higher levels of PC-rich lecithin help to ensure stability and more comprehensive encapsulation.

B. Mixing of total compound in smaller containers, thereby allowing more thorough and uniform blending. This is as opposed to typical mixing on larger scales, which hampers proper fluidization.

C. Part of the total methodology of this invention requires pre-treatment of nutrients to be encapsulated. This can include, but is not limited to, wet milling, or partial dissolution using low or high shear wet milling (depending on substrate to be milled), to make active ingredients uniformly smaller and more accepting of the invention's encapsulation. This method also protects the integrity of the active compound being treated.

D. Other important reasons for mineralizing the water are increasing Zeta Potential and improving stability. Typical water used in pharmaceutical/nutraceutical manufacturing is distilled through de-ionization or reverse osmosis. This form of water, while pure, typically has aggressive receptor properties vs. aggressive donor properties. As a “receptor,” it can become acidified by complexing with CO₂ (for example) as well. Empty, aggressive reception, and/or acidified water can disrupt surrounding mediums, including aqueous mediums containing nutrients. By aggressively mixing the water in a consistent vertical motion, the water becomes more structured. This motion also stabilizes the water portion of the liposomal sphere with added electrolytes, which causes the water to become more biocompatible, stable, and less disruptive to the nutrients contained therein. Therefore the entire final prodosome structure is more stable.

E. The invention starts with pharmaceutical grade water to ensure purity, and then adds a precise pre-measured amount of mineral electrolytes at the appropriate time to “mineralize” the water as just indicated above. This process ensures uniformity of mineral levels and distribution during each production process and also ensures a finished compound that has more of the biocompatible properties of body fluids and more readily promotes competent cell metabolism. Also, unlike relying on mineral water from a natural source, which can have impurities, varying potencies of minerals, and a complete absence of one or more mineral compounds, the process of the present invention ensures that the mineral electrolytes are supplied in uniform, ample, and comprehensive amounts. To this point, a 30-50 gallon batch of finished product was allowed to sit for 7 hours and experienced an exothermic reaction in which the temperature of the batched product rose up to 98.6 Fahrenheit, i.e., the temperature of body fluids, and then stopped. The present invention is creating a specific resonance that is completely biocompatible with body fluids.

F. The invention's inclusion of trace minerals contributes to intracellular pH regulation and homeostasis and pH stability in the liposomal sphere contained within the product prodosome, especially important because enveloped nutrients (e.g., Vitamin C) may disrupt pH balance. By avoiding this circumstance, additional stability is provided for the liposomal sphere contained within the product prodosome. Furthermore, the ability of the SK713 liposomal sphere contained within the product prodosome, infused and saturated with our special mineral rich electrolyte material, is that the sphere can impart, through the action of mineral buffering, a pH balancing effect within the bloodstream concurrently with the release of the contained nutrients. It should not be inferred that the pH of the SK713 or its substrate impose any buffering effects because of their pH properties. Rather, the SK713 and the ionic constituents contribute buffering potential as need for the body's homeostatic requirements. This phenomenon can improve cellular uptake and utilization of available nutrients.

Other known liposomal technologies are plagued with instability; gradual and continual degradation of liposomal capsules; and substrate “leakage” out of degrading and delineating liposomes ultimately results in a reduction and eventual loss of liposomal encapsulating benefits. Evidence of this degradation are visible in product containers as solid residues continue to amass, precipitate, and accumulate on the bottom of the containers. In contrast, thoroughly and completely “prodosomed” product remains completely and evenly dispersed and homogenized throughout the blended mixture. The SK713 process helps to ensure that capsule stability, homogeneity, and therefore stronger and more sustained benefits, occur from products treated with prodosomes in embodiments of the present invention.

Surface Tension Measurement

Other beneficial properties are evidenced by the Surface Tension testing done on standard liposomes vs. Prodosomes as prepared in Example 1. Testing was performed by NSL Analytical. Two liquid samples were submitted for Contact Angle measurement on a glass slide surface. The test outlined was performed on both samples. The measurements were recorded at five second intervals due to the small area of contact. Once the drop (10 μL) was in contact with the surface, the first measurement was recorded, and the second measurement was recorded after approximately five seconds, and the same for the third, fourth, and fifth. Sample #1 (standard liposome) demonstrated an average Contact Angle of 39. Sample #2 (Prodosome) demonstrated an average Contact Angle of 47.7. The inclusion and specific mixing process of the trace minerals into the Prodosomes increased the average level of surface tension by 22.3%. The increased surface tension has a direct and significant impact on liposomal integrity and can be attributed to the SK713 Process, which as previously discussed, increases Zeta Potential, thereby reducing agglomeration and increasing the dispersion and subsequent stability of the solution. A higher Zeta Potential leads to a stronger level of electrostatic repulsion within the solution and subsequent stronger liposomal shell(s) in the clustoidal multilamellar SLP prodosome structure of Example 1.

Advantages produced by this process include increased stability of the liposomal transport sphere contained within the product prodosome while simultaneously not adding to the cost or burden of producing the material. It also affords an increased opportunity to enhance cellular uptake of nutrients, both by balancing extracellular and intracellular pH and by bolstering extra- and intracellular fluid exchange. These actions occur concurrently with the delivery of nutrients, which creates additional synergies to benefit health. A replenishment of electrolytes is vital to maintaining a balanced osmotic gradient within plasma to ensure optimal oxygenation and correct hydration via maintaining optimum pH. It is this correct hydration and pH that affects all other usage of nutrients delivered by the liposomal sphere contained within the product prodosome.

In embodiments of the present invention, the process as described herein is focused on a new paradigm of altering the functionality of the liposome, giving it a dual purpose. With the SK713 Prodosome, the liposome now acts as both a delivery vehicle and a functional enhancer of the receptor or target of the delivered materials.

Other advantages also include low cost of production; ease of transport for usage on site; no additional or unusual equipment needed for usage; able to be stored at room temperature; better stability of SK713 material and better stability of liposomal material containing enveloped nutrients within the product prodosome; process uses pre-preparation of active ingredients to be Prodosomed in order to ensure better and more thorough encapsulation; and the creation of electrically charged, energy-enhanced phospholipids of the Prodosome that acts as a transport vehicle while also actively influencing cellular integrity for enhanced utilization of nutrients.

### EXAMPLE 2

Effect of SK713 SLP Encapsulated VMP35 Multivitamin Formulations (Prodovite®) on Human Blood

Experimental Design

SK713 SLP encapsulated VMP35 MNC formulation was prepared using the method described in Example 1. This example relates to a controlled cross-over study to evaluate the effects of transmucosal administration of SK713 SLP encapsulated VMP35 MNC (active) as opposed to baseline and commercially available bottled water (control). Thirty-eight (38) subjects were recruited from random interviews. There were ten (10) males and twenty-eight (28) females ranging in age from twelve (12) years to eighty-two (82) years with an average age for males of forty-nine (49) years and for females of forty-six point eight (46.8) years as shown in Table 8. Subjects were assigned randomly into one of three groups (baseline, control, and active) and underwent peripheral blood smear (“PBS”) live blood cell imaging (“LBCI”) as shown in Table 9. The baseline blood samples were drawn from all of the subjects prior to transmucosal administration of VMP35 MNC formulation or transmucosal administration of water to the same subjects. Changes in peripheral blood smear (PBS) were examined using Live Blood Cell Imaging and Phase Contrast Microscopy. See, e.g., G. Popescu, et al., Imaging red blood cell dynamics by quantitative phase microscopy, 41 BLOOD CELLS, MOLECULES & DISEASES 10 (2008), incorporated by reference herein in its entirety.

TABLE 8
Randomly Selected Subjects Participating
in Live Blood Cell Imaging

| | | | | Self-Reported |
|---|---|---|---|---|
| Participant | Age | Gender | Ethnicity | Health Issues |
| 10 | 37 | Female | Guyanese | None |
| 11 | 45 | Male | Caucasian | High Blood |
| | | | | Pressure (“BP”) |
| 12 | 16 | Female | Caucasian | None |
| 13 | 13 | Female | Caucasian | None |
| 14 | 37 | Female | Caucasian | Allergies |
| 15 | 43 | Female | Caucasian | Poor Digestion |
| 17 | 70 | Female | Italian | Osteoporosis, |
| | | | | Arthritis |
| 18 | 24 | Male | Lebanese | None |
| 19 | 22 | Female | Caucasian | None |
| 20 | 22 | Female | Caucasian | None |
| 21 | 61 | Male | Caucasian | None |
| 22 | 51 | Female | Caucasian | None |
| 23 | 37 | Male | Caucasian | None |
| 24 | 62 | Female | Caucasian | Skin Condition |
| 25 | 54 | Female | Caucasian | None |
| 26 | 63 | Female | Caucasian | Diabetes |
| 27 | 58 | Female | Caucasian | None |
| 28 | 43 | Male | Caucasian | Digestion |
| | | | | Problems |
| 29 | 49 | Female | Caucasian | None |
| 30 | 51 | Female | Caucasian | None |
| 31 | 24 | Female | Caucasian | Attention Deficit |
| | | | | Disorder |
| 32 | 61 | Female | Caucasian | Thyroid, Severe |
| | | | | Pain |
| 33 | 56 | Female | Caucasian | None |
| 34 | 60 | Female | Caucasian | None |
| 35 | 58 | Female | Caucasian | Depression, |
| | | | | Thyroid, |
| | | | | Hormone |
| 36 | 79 | Female | Caucasian | High BP, |
| | | | | Diabetes, Heart |
| 37 | 35 | Female | Caucasian | None |
| 38 | 40 | Female | Caucasian | None |
| 39 | 57 | Female | Caucasian | None |
| 40 | 12 | Female | Caucasian | Skin Condition |
| 44 | 44 | Male | Trinidadian | None |
| 45 | 50 | Male | Italian | None |
| 46 | 50 | Female | Caucasian | Toxic Exposure |
| 47 | 71 | Male | Caucasian | Severe |
| | | | | Periodontal |
| | | | | Disease |
| 48 | 74 | Female | Italian | High Blood |
| | | | | Pressure |
| 49 | 82 | Male | Italian | Bladder Cancer, |
| | | | | Cl1 |
| 50 | 33 | Male | Caucasian | Herpes |
| 51 | 56 | Female | Italian | None |

TABLE 9
Group of Subjects and Blood Test Time

| | | 5 minutes | 5 minutes | 30 minutes |
|---|---|---|---|---|
| | | after | after | after |
| Groups(n) | Baseline | water | VMP35 MNC | VMP35 MNC |
| Group 1 | Group 1 | Group 1 | Group 1 | |
| (n = 8) | | (control) | (active) | |
| Group 2 | Group 2 | | Group 2 | |
| (n = 23) | | | (active) | |
| Group 3 | Group 3 | | Group 3 | Group 3 |
| (n = 7) | | | (active) | (active) |
| Total tests | 38 | 8 | 38 | 7 |

After taking baseline blood samples, PBS Group 1 (n=8) consumed 30 mL water with a follow-up PBS taken at 5 minutes. The moment of administration of water or VMP35 MNC formulation to a subject is used at time zero. Both active groups, Group 2 (n=26) and Group 3 (n=7) consumed 30 mL of VMP35 MNC with a follow-up PBS taken at 5 minutes. Thereafter, Group 3 had an additional PBS taken at 30 minutes. Group 1 then consumed 30 mL of VMP35 MNC and had a PBS at 5 minutes after intake. The dosing regimen and sampling schedule are summarized below.

Group 1: Water Control group consisting of 8 individuals (3 blood samples each):

a. Baseline blood test prior to the intake of water

b. 2nd blood test at 5 minutes after the intake of water

c. 3rd blood test at 5 minutes after the administration of VMP35 MNC

Group 2: Active Group consisting of 23 individuals (2 blood samples each):

a. Baseline blood test prior to the administration of VMP35 MNC

b. 2nd blood test at 5 minutes after the administration of VMP35 MNC

Group 3: Active Group consisting of 7 individuals (3 blood samples each):

a. Baseline blood test prior to the administration of VMP35 MNC

b. 2nd blood test at 5 minutes after the administration of VMP35 MNC

c. 3rd blood Test at 30 minutes after the administration of VMP35 MNC

Results

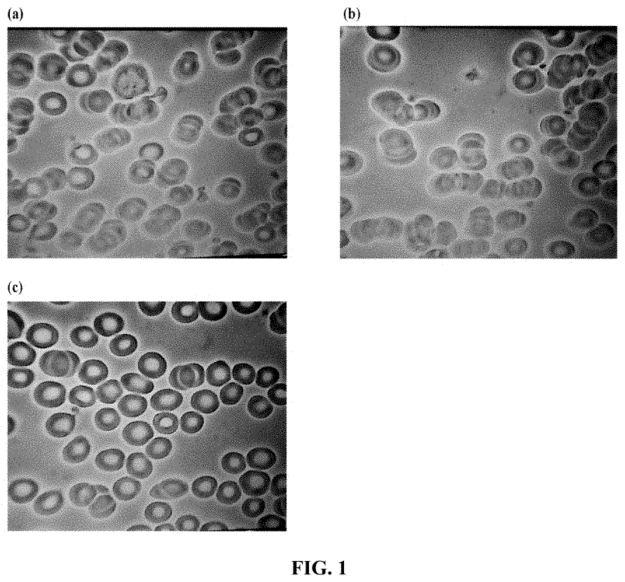

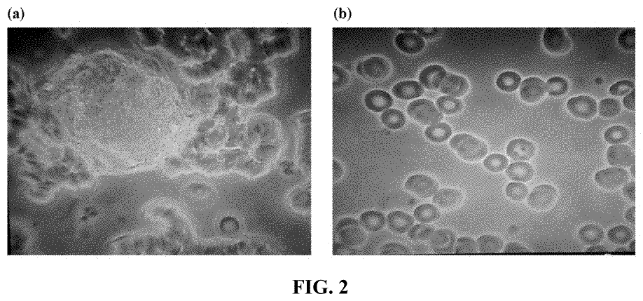

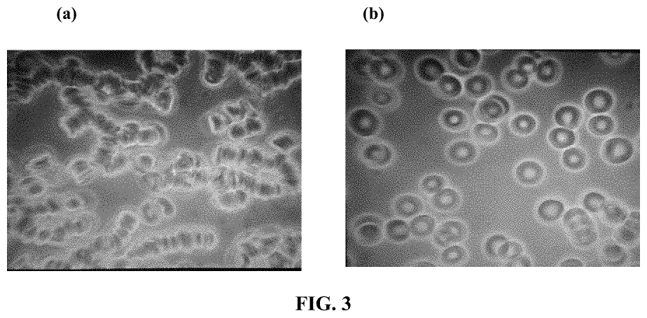

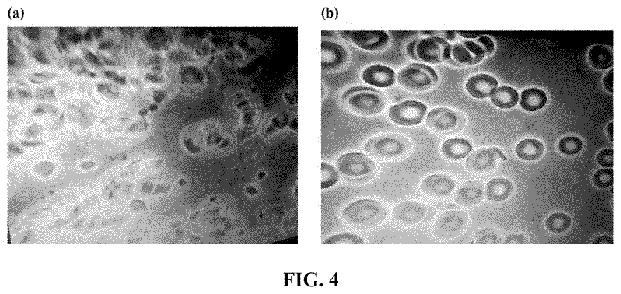

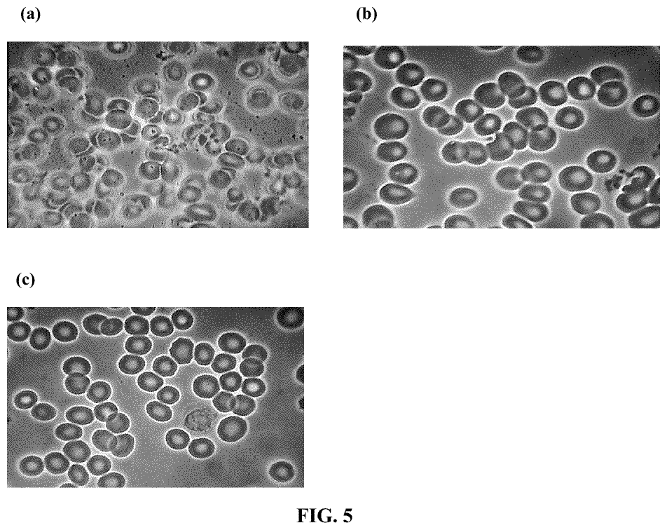

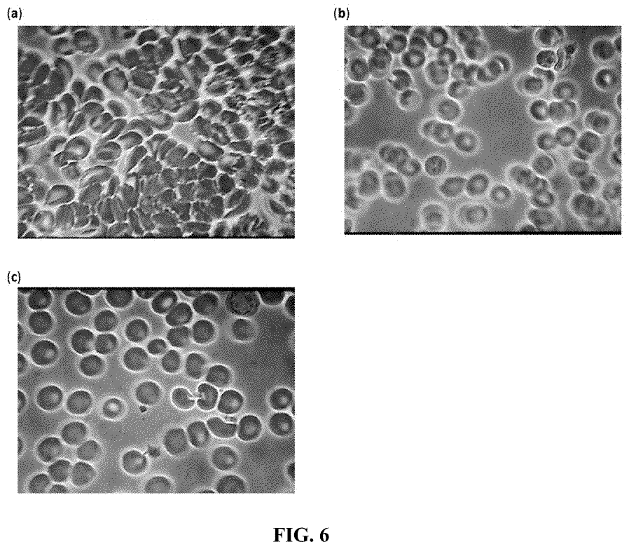

A non-blinded comparison was done between the baseline and subsequent PBS samples. Pictures were taken for blood samples during each phase of the study. For each group, changes in morphological, hematological, and rheological characteristics were recorded. Representative results are depicted in FIGS. 1-6. Specifically, FIGS. 1(a) and (b) indicate that no changes were observed between the baseline and the 5-minute samples in the control group (Group 1). Substantial differences were observed between the baseline and 5-minute samples in the active Groups 1 and 2. (See FIGS. 1(b) and (c), FIGS. 2(a) and (b), FIGS. 3(a) and (b), and FIGS. 4(a) and (b)). Substantial differences were observed among the baseline, 5-minute, and 30-minute in the active Group 3. (See FIGS. 5(a), (b) and (c), and FIGS. 6(a), (b) and (c)). Improvements in the splayed arrangement, size, form, density, and distribution of RBCs following intake of the VMP35 MNC can be clearly identified in these figures and are indicative of improved morphological, hematological, and rheological properties.

Baseline and Control

Images of red blood cells (RBCs) obtained from baseline and the 5-minute samples in the control group clearly showed aggregation and immobility—a sludge effect, malformation and damage, and extensive hypochromic state (i.e., an oversized “donut hole” evidencing reduced hemoglobin). In the images of baseline samples, protoplasts (a biomarker associated with increased acid burden), extensive “debris” in the plasma, and “dwarfed” white blood cells (“WBCs”) were also observed.

RBC Improvements 5 Minutes after the Administration of VMP35 MNC

RBC improvements 5 minutes after the administration of VMP35 MNC (shown in FIGS. 1-6) included a breakup of aggregation and splaying out of RBCs on the slide, improvement in spherical formation of RBC, and a progressive reduction (with time) of hypochromicity. Other positive effect of transmucosal VMP35 MNC included improved movement and ability to flow (rheology) of RBCs in the plasma, evidencing improved hydration, reduced viscosity, and reduced surface tension.

RBC Improvements 30 Minutes after the Administration of VMP35 MNC

LBCI results of Group 3 at 5 minutes and 30 minutes post intake of VMP35 MNC shown in FIGS. 5 and 6) were similar to each other, both of which showed improved hemoglobin concentration, a reduction in plasma debris (cleaner plasma), and reduced quantity of protoplasts.

Overall, RBC and blood rheology improvements observed in this example demonstrate that SK713 encapsulated VMP35 MNC formulation can be absorbed and delivered to the blood within 5 minutes through sublingual transmucosal administration. The central finding of this example is the fact that the improvements occurred within 5 minutes after the administration of VMP35 MNC formulation and were sustained for at least 30 minutes. Conversely, no such changes were found when the equivalent volume of water was ingested by the control group, which adds credibility to the baseline findings and demonstrates reproducibility in the absence of active intervention. ON the other hand, the prompt, sustained and progressive findings in Group 2 at 5 minutes and Group 3 at 5 and 30 minutes offer support that the observations were also valid metrics to observe the bioactive effects. This conclusion is further strengthened by the appearance of the same results in Group 1 during the active cross-over phase (switching to VMP35 MNC formulation).

This example demonstrates that the SK713 SLP delivery technology exerts rapid positive effects on morphological, hematological, and rheological properties of the blood. This rapid response also suggests that the SK713 SLP technology efficiently delivers nutrients into the blood via the sublingual mucosa, in less than 5 minutes from intake and may overcome digestive inefficiencies in vivo.

### EXAMPLE 3

Effect of SK713 SLP Encapsulated VMP35 Multivitamin Formulations on Cytoprotection in Human Subjects

This study evaluated the absorption rate of the iron-free VMP35 and its effects on live human blood by assessing the changes in peripheral blood smears (PBS) from baseline (0 min) incorporating live blood cell imaging (LBCI) using phase contrast microscopy [Olympus BX-30 light microscope equipped with a phase contrast condenser (Tokyo, Japan) in conjunction with a 150-W lightbox and fiber-optic cable assembly] at 5-min post-control intake and 30-min post-VMP35 intake, respectively. It is important to indicate that the lens configuration was adjusted using a 10× eyepiece and 100×-oil immersion objective magnification to approximately ascertain a 1000-× magnification. Specifically, the lighting generated a superior level of cell definition, brightness, clear morphology, and can clearly and distinctly features of the cell membranes.

Significant efficacy of the iron-free VMP35 was observed on hemoglobinization, blood oxygenation, hydration, and neutrophil morphology at 5- and 30-min following a baseline evaluation, respectively. In fact, VMP35 instantly enhanced the morphological, hematological, and rheological properties of live human blood, and it can be concluded that the iron-free VMP35 produced adequate nutritional benefits to restore intracellular iron-dependent RBC hemoglobin within 5 min of intake, which was further sustained for an extended period. In addition, neutrophil white blood cells demonstrated dramatic improvement in numbers and morphology. No adverse events were observed.

Materials and Methods

SK713 Soy Lecithin Phospholipid (SLP)-encapsulated VMP35 MNC nutraceutical formulation) (Prodovite®: The above-described Prodosome encapsulation technology was used in a state-of-the-art multi-step cGMP- and NSF-certified manufacturing process to develop this unique nutraceutical formulation. The product manufacturing batch/lot number is 13070-2 (Mfg. Date, Mar. 22, 2013). The first step comprises the production of SK713 SLP, which uses a minimum of 85% non-GMO phosphatidylcholine and its impregnation and saturation with solar-dried electrolytes to ensure the supply of free ions to amplify the ionic properties on each level of the multi-lamellar phospholipid spheres. The second step utilizes a progressive high-shear wet milling pre-treatment of the active ingredients including structurally diverse antioxidants, multivitamins, micronutrients, minerals, and standardized botanical phytonutrients. The next step involves a specific blending, impregnation, and encapsulation technology to achieve a novel, multi-lamellar, energetically enhanced, liposomal, encapsulated supplement (Prodovite®). Thus, the constituents in the VMP35 liquid supplement (Prodovite®) are encapsulated in SK713 SLP multi-lamellar, custodial, non-GMO phospholipid, prodosomal, nutrient-absorption technology.

Study Participants: A total of 38 subjects [Men=11; Women=27; Ages: 22-82 years] were randomly recruited at random from medical health clinics during interviews in Woodbridge and Perth, Ontario, Canada. Necessary institutional review board (IRB) approval was obtained. (See Table 10.

TABLE 10
Study Participants, Age, and Health Problems

| | | | | Self-Reported |
|---|---|---|---|---|
| Participant | Age | Gender | Ethnicity | Health Issues |
| 10 | 37 | Female | Guyanese | None Reported |
| 11 | 45 | Male | Canadian | High Blood |
| | | | | Pressure (“HBP”) |
| 12 | 37 | Female | Canadian | None Reported |
| 13 | 43 | Female | Canadian | Poor Digestion |
| 14 | 70 | Female | Italian | Osteoarthritis, |
| | | | | Osteoporosis |
| 15 | 24 | Male | Lebanese | None Reported |
| 16 | 22 | Female | Canadian | None Reported |
| 17 | 22 | Female | Canadian | None Reported |
| 18 | 61 | Male | Canadian | None Reported |
| 19 | 51 | Female | Canadian | None Reported |
| 20 | 37 | Male | Canadian | None Reported |
| 21 | 62 | Female | Canadian | Skin Condition |
| 22 | 54 | Female | Canadian | None Reported |
| 23 | 63 | Female | Canadian | Diabetes |
| 24 | 58 | Female | Canadian | None Reported |
| 25 | 43 | Male | Canadian | Digestion |
| | | | | Problem |
| 26 | 49 | Female | British | None Reported |
| 27 | 51 | Female | Canadian | None Reported |
| 28 | 24 | Female | Canadian | Attention Deficit |
| | | | | Disorder |
| | | | | (“ADD”) |
| 29 | 61 | Female | British | Thyroid, Severe |
| | | | | Pain |
| 30 | 56 | Female | Canadian | None Reported |
| 31 | 60 | Female | British | None Reported |
| 32 | 58 | Female | British | Depression, |
| | | | | Thyroid, |
| | | | | Hormone |
| 33 | 79 | Female | Canadian | HBP, Diabetes, |
| | | | | Heart Problems |
| 34 | 35 | Female | Canadian | None Reported |
| 35 | 40 | Female | Canadian | None Reported |
| 36 | 57 | Female | Canadian | None Reported |
| 37 | 40 | Female | Canadian | Depression |
| 38 | 30 | Male | Canadian | None Reported |
| 39 | 70 | Female | Canadian | None Reported |
| 40 | 44 | Male | Trinidadian | None Reported |
| 41 | 50 | Male | Italian | None Reported |
| 42 | 50 | Female | Canadian | Toxic Exposure |
| 43 | 71 | Male | British | Severe |
| | | | | Periodontal |
| | | | | Disease |
| 44 | 74 | Female | Italian | HBP |
| 45 | 82 | Male | Italian | Bladder Cancer, |
| | | | | Chronic |
| | | | | Lymphocytic |
| | | | | Leukemia |
| | | | | (“CLL”) |
| 46 | 33 | Male | Canadian | Herpes |
| 47 | 56 | Female | Italian | None Reported |

Live Blood Cell Imaging in this sample population demonstrated that even in “None Reported” subjects, widespread immunological challenges are evidenced by unanimous and significant cell aggregation and poor blood cell rheology.

Study Design: This observational, pilot, controlled 1-way crossover study was designed to evaluate the effect of a non-iron containing (i.e. iron-free) VMP35 liquid multi-nutrient complex (MNC) (“Treatment Group”) on blood oxygenation and hydration compared to the placebo group (“Control”) at Baseline, 5 Minutes, and 30 Minutes post-treatment, respectively. In this study a 1 fl. oz. dose was swished in the mouth for 30 seconds and then swallowed This clinical evaluation investigated the rate of absorption as assessed by effects on various properties of live human blood induced by the orally consumed VMP35 liquid nutraceutical supplement. Changes in peripheral blood smears from Baseline (0 minutes) were observed using live blood cell imaging (“LBCI”) with phase contrast microscopy at 5 minutes post-control intake, and 5 minutes and 30 minutes post-supplement intake.

Live Cell Imaging: LBCI was conducted by Veritas Health Inc. (Woodbridge, Ontario, Canada) using an Olympus BX-30 light microscope equipped with a Phase Contrast Condenser (Tokyo, Japan). A 150-W lightbox with fiberoptic cable assembly was used to highlight the specimen against a gray field, and increase the range of intermediate shades. The lighting produces a high level of cell definition, clear morphology, and can distinguish features of some cell membranes. The lens configuration was 10× eyepiece and 100×-oil immersion objective magnification, to achieve approximately 1000× magnification. Oil immersion achieved finer resolution and brightness.

Blood Smear Handling: Peripheral blood smear (PBS) was obtained from the finger using a Bayer Single-Let Disposable Lancet 23G 2.25-millimeter sterile single-use lancing device (Whippany, New Jersey, United States). Being careful not to squeeze the finger, a small amount of capillary blood was expressed due to capillary pump action, collected and transferred directly onto microscope slides. The slides used were pre-cleaned using standard 1-inch by 3-inches with a thickness of 1-millimeter supplied by Electron Microscopy Sciences (Hatfield, Pennsylvania, United States). The slide was covered quickly and gently with a cover glass without pressure to protect blood cells from damage. The cover glass was pre-cleaned #1 22 mm×40 mm with 0.13- to 0.17-mm thickness supplied by Electron Microscopy Sciences (Hatfield, Pa., United States). The corners of the cover glass were tapped carefully to disperse surface tension and create an even layer for viewing. The slide was then transferred directly to the microscope for viewing. Evaluation of blood properties began in less than 30 seconds after the blood samples were collected. Consistent blood extraction and handling procedures were followed to avoid artifacts. A peripheral blood smear was drawn to assess both the placebo and treatment samples over a period of 0, 5, and 30 minutes post-treatment.

Adverse Events: A large number of participants upon entry into the study were suffering from an array of chronic diseases including anemia. Adverse events were strictly monitored.

Case Study Report: An independent case study report was added with proper consent from the patient who had a stroke in early 2018. The patient took 2- to 3-ounces of Prodovite® VMP35 per day over a period of a little over 6 months. The time-dependent improvement has been included in the Results section.

Results

The findings of the study were very encouraging. Significant improvement in blood properties, including hemoglobinization, in the Treatment Group was observed following treatment with Prodosome-encapsulated non-iron VMP35 MNC nutraceutical formulation.

Control Group: No changes were observed between the baseline and 5-minute samples.

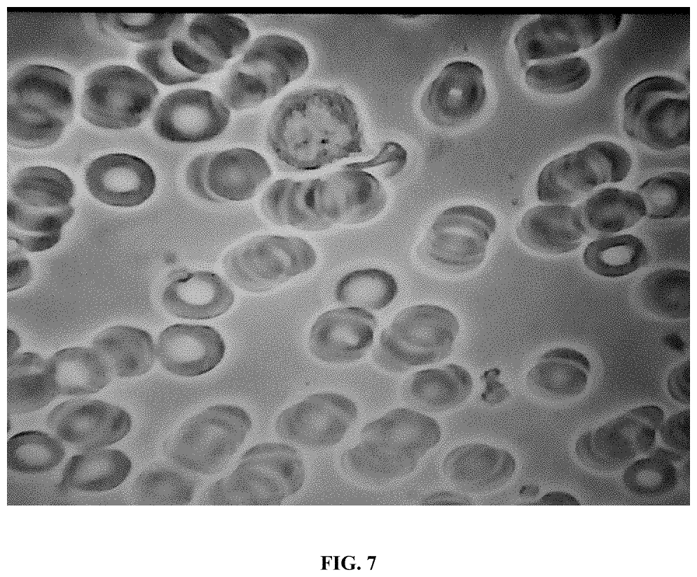

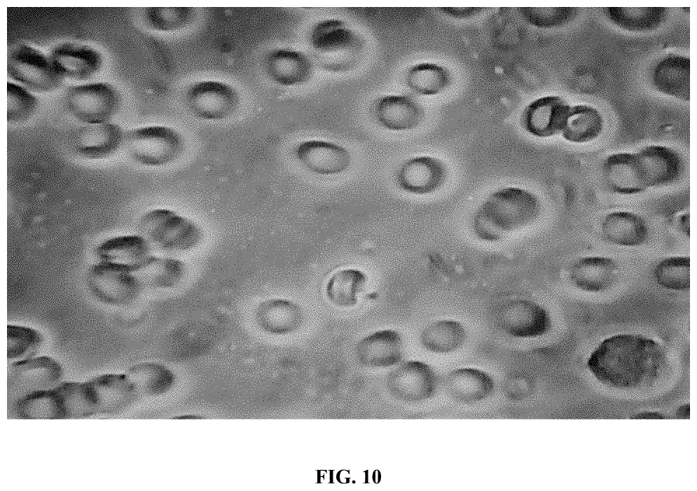

Treatment Groups: Substantial differences were observed between the baseline and 5-minutes after Prodovite® VMP35 supplementation. Group 1: FIGS. 7 (baseline before water intake), 8 (5 minutes after water intake), and 9 (5 minutes after VMP35 intake); and Group 2: FIGS. 10 (baseline before VMP35 supplementation), and 11 (5 minutes after VMP35 supplementation).

30-Minute Post-Treatment Group: Substantial differences were observed between the baseline, 5- and 30-mintues post-active groups. Group 3: FIGS. 12 (baseline before VMP35 supplementation), 13 (5 minutes after VMP35 supplementatino), and 14 (30 minutes after VMP35 supplementation). Following are the itemized observations in the baseline, post-supplement red blood cell (“RBC”) improvements after 5 minutes and after 30 minutes, respectively.

Baseline: Observations in the Control Group and 5-minute post-treatment samples including aggregation and immobility—a sludge effect; malformation, damage, and extensive hypo-chromic state (i.e., an oversized “donut hole” evidencing reduced hemoglobin). At baseline, extensive “debris” in the plasma and “dwarfed” white blood cells were also observed. A cross-section of ages and a variety of conditions were represented by the subjects so that the individual baseline peripheral blood smear examples shown in the figures do not look similar.

Post-Supplement Red Blood Cell (RBC) Infrastructural Improvements After 5 Minutes Post-Treatment: Post-supplementation RBC improvements after 5 minutes included a breakup of aggregation and splaying out of RBCs on the slide, demonstrating reduced viscosity; improved RBC spherical formation and a progressive reduction (with time) of hypochromicity. Other post supplementation observations include improved movement and ability to flow (rheology) of RBCs in plasma, evidence of improved hydration, reduced viscosity, and reduced surface tension.

Post-Supplement RBC Infrastructural Improvements After 30 Minutes Post-Treatment: Group 3 samples were valuated at 5 and 30 minutes post-intake of the VMP35 and similarly showed marked improvement in biomarkers from baseline. Observations of supplementation post-intake, at 5 and 30 minutes, also showed reduced or eliminated hypochromicity, interpreted as improved hemoglobin concentration, and a reduction in plasma debris. The plasma appeared cleaner, possibly due to a reduced quantity of sequestrants (of unknown origin), which are greater in number in blood with greater cellular aggregation and reduced hydration. Overall, improvements in the splayed arrangement, size, form, density, and distribution of RBCs following the intake of the VMP35 were observed.

Taken together, VMP35 caused dramatic morphological, hematological (including restoration of hemoglobin and neutrophils), and rheological changes in the blood following 5 minutes of administration, which were sustained for at least 30 minutes. Improved blood rheology was observed by videographic assessment of live red blood cell (RBC) movement on microscope slide, which is represented in still photographs by a significant reduction in RBC aggregation, improved RBC morphology, and distribution. Overall, VMP35-induced rapid movements in blood properties, restored red blood cell (RBC) hemoglobin saturation and morphology, and improved neutrophil morphology within 5 minutes that were sustained for 30 minutes post-supplement intake. Following are the salient findings:

RBC and blood rheology improvements were observed demonstrating that VMP35 was delivered and absorbed by sublingual trans-mucosa within 5 minutes;

VMP35 exhibited a unique ability to initiate rapid onset of hematological changes in response to intervention;

Changes were observed within 5 minutes of VMP35 administration, which were sustained for at least 30 minutes. Group 2, at 5 minutes, and Group 3, at 5 and 30 minutes, support the validity of the observations. No effect was observed in the control group. Prompt, sustained, and progressive results were observed in the Treatment Groups;

Overall, VMP35 exerted a rapid, positive response on morphological, hematological, and rheological properties of the blood.

Adverse Events Monitoring: No significant adverse events were observed. A significant number of subjects were already suffering from an array of chronic disorders upon entry into the study. However, no treatment-related adverse events were noted in either placebo or treatment groups.

### EXAMPLE 4

Case Study Report

A recent Case Study was reported using the oral supplementation of Prodovite® VMP35 over a period of slightly more than six consecutive months in a subject in Norwich, N.Y., United States.

Physicians Involved: Dr. Piotr Sadej, M.D.; Dr. Sundar Jayaraman, M.D.; Dr. Karen R. Banks-Lindner, D O, FLLC. The detailed Doctor Case Report was provided with permission by Mr. David J. Evans.

Subject: A 56-year-old Caucasian male stroke (cerebral infarction) patient.

Following is the Case History, Treatment Profile, Regimen, and Consequential Events:

Jan. 5, 2018: Patient had a multi-planar multi-sequence MM of the brain (on a 1.5 T MRI system) as a follow-up of a CT done the previous day owing to a stroke (right-sided weakness, and hypertension). The MRI revealed a left para-median distribution infarct specifically involving the pons and the medulla. Chronic lacunar infarcts and possible subacute white matter infarcts were also seen. These chronic changes were seen in the setting of white matter disease that could relate to microangiopathy. Multiple tiny foci of iron-containing hemosiderin evident in bilateral thalami, basal ganglia, brainstem, cortical/subcortical regions, and cerebellum.

CBC 5 Weeks (mid-February 2018) Post Stroke:

Red Blood Cells: 4.42 (normal range being 4.0 to 5.8)

Hemoglobin: 2.8 gm/dL (normal range being 13.0 to 18.0); indicates anemia

Hematocrit: 38.1 (normal range being 37.0 to 52.0); low normal evidence of anemia

Platelet Count: 172 (normal range [in thousands] being 150 to 450); low normal can indicate a trend towards an anemic condition

Red Blood Cell Distribution Width (“RDW”): 15.8 (normal range being 11.5 to 14.0); a higher number indicates probable IDA

Patient started consuming from about 2 to about 3 ounces (from about 56.7 to about 85.0 grams) per day of Prodovite® VMP35 from the middle of May 2018.

May 2018 Test Results

Creatinine: 1.5 mg/dL (normal range being 0.5 to 1.2) indicates kidney challenges

Glucose: 105 mg/dL (normal high limit being 99); blood sugar is high

August 2018 Test Results

Red Blood Cells: 4.59 (4.0 to 5.8); mild improvement

Hemoglobin: 14.4 gm/dL (normal range being 13.0 to 18.0); significant improvement

Hematocrit: 42.3 (normal range being 37.0 to 52.0); improved

Platelet Count: Not Available

RDW: 12.6 (normal range being 11.5 to 14.0); significant improvement

November 2018 Test Results

Red Blood Cells: 5.01 (normal range being 4.0 to 5.8); improved—normal

Hemoglobin: 15.6 gm/dL (normal range being 13.0 to 18.0); improved—normal

Hematocrit: 45.9 (normal range being 37.0 to 52.0); improved—normal

Platelet Count: 202 (normal range [in thousands] being 150 to 450); trending improvement

RDW: 12.9 (normal range being 11.5 to 14.0); improved—normal

Consequential Physician's Evaluation and Findings on Dec. 6, 2018

A follow-up brain MRI was done on Dec. 16, 2018 (owing to the history of cerebral infarction and the patient's right hemiparesis and gait disturbance). This study was compared to the previous brain MRI from Jan. 5, 2018. No acute infarction was observed. Sequela of previous infarction of the lower pons and upper medulla seen (pontine and cerebellar encephalomalacia) along with evidence of previous hypertensive microhemorrhages (microscope changes in the cerebral white matter).

Along with the above MM, a magnetic resonance angiogram (“MRA”) was done at the same time with the following findings: there was no occlusion or hemodynamically significant stenosis of major intracranial arteries.

Overall Conclusion: Non-iron containing Prodovitex® VMP35 has the ability to significantly improve hematological characteristics in the subject.

In the following Examples, an additional athletic case study and two concept validation Pilot Studies were conducted in a diverse population of well-trained athletes to assess and confirm the effects of the World Anti-Doping Association (‘WADA’) compliant iron-free VMP35 on athletic performance.

### EXAMPLE 5

Athletic Case Study

The subject was a well-trained male athlete specializing in power lifting, certified in sports nutrition and personal training (AAAI/ISMA). Subject has used a very extensive supplement regimen to support his intense power lifting workouts over the previous 12 months of serious strength training. His personal best in squat weightlifting was 395 lbs. He was attempting to achieve a new personal record of 405 lbs. Prior to supplementing with the VMP35 MNC, to ensure his system was totally clean from any supplement influence, he did a 6-week washout and stopped taking the 20 other supplements his research indicated he should be using up to that time. Shortly before starting an intense Powerlifting workout session, he took 1-ounce of iron-free liquid VMP35, swishing it in his mouth 30 seconds before swallowing. He also added 1-ounce of VMP35 to his regular workout beverage and sipped on it between his sets of squats, swishing it each time briefly before swallowing. He ended his first workout session by achieving a squat lift of 515 lbs (and contrary to peer warnings, the following day he had no muscle pain). In the following 2 weeks, he continued to experience strength increases in squat training exercises ranging from 545 lbs to 575 lbs (a 180 lb increase over the 395 lb personal best at baseline).

### EXAMPLE 6

Concept Validation Pilot Studies

To confirm the veracity of the previous athletic case study, conducted a 15-day concept validation pilot clinical investigation was conducted in three healthy young male athletes (age: 32-36 years), on the effects of WADA compliant iron-free liquid VMP35 in well trained athletes. Duly signed Informed Consent Forms were obtained from the study participants. Regulatory approvals were obtained and adverse events were critically monitored. The daily dose was 1-ounce consumed BID and swished in mouth for 30 seconds before swallowing. The first dose was about 20 to 30 minutes before engaging in an extremely rigorous exercise regimen. The second dose was consumed later that day. Over the course of the 15-day study, the most significant improvements were experienced within the first two days.

Subject 1. Before VMP35 supplementation, the first subject (male 36 years old) was struggling with 270 lbs. for 4 reps on the Hack Squat. After VMP35 supplementation, subject achieved a squat of 270 lbs. for 10 reps; rested, then the very next set increased to 320 lbs. for 10 reps. The 3rd and final set of that exercise, he increased the weight again to 360 lbs. for 8 reps.

Furthermore, another increase in strength was experienced in banded hammer strength incline press. Pre-PV, Subject was pressing 180 lbs. for 10 reps, which was increased to 230 lbs. for 10 reps. Moreover, following VMP35 supplementation, respiratory capacity significantly increased.

Subject 2. On the banded reverse hack squat, Subject #2 (male, 32 years old) experienced a significant increase in strength. Pre-VMP35, he achieved a weight of 160 lbs. for one set of 8 reps. Post-VMP35 intake he increased to 180 lbs. for 2 sets of 10 reps.

On the Hammer Strength banded incline chest press, Pre-VMP35, subject's working weight sets were 160 lbs. Post-VMP35, his sets increased to 180 lbs. On side lateral dumbbell raises, Subject's working weight increased from 20 lbs. Pre-VMP35 to 25 lbs. Post-VMP35.

Subject 3. Pre-VMP35 Supplementation, the basic squat result for Subject #3 (male, 36 years old) was 405 lbs. for 10 Reps. Post VMP35, his squat weight increased significantly to 455 for 6 reps. He experienced an increase in muscle mass after VMP35 supplementation; a shorter recovery time between sets and after workout. Over the course of the 15 day study, Subject #3 also reported enhanced sleep quality, increased appetite, and consistently increased overall energy levels.

### EXAMPLE 7

A Pilot study was carried out to evaluate the effects of the WADA compliant iron-free liquid VMP35 supplement in trained cyclists to confirm the beneficial effects across a range of athletic endeavors.

Athletic Cyclists Case Study

Subject 1. A 47-year old male cyclist consumed 1 fl. oz. BID of VMP35 for 2-consecutive weeks. His power output (W) improved from 317 to 325.5 (a 2.7% increase), and his heart rate increased by only 1%.

Subject 2. In another subject, a 52-year old male cyclist, who consumed 1 fl. oz. BID of VMP35 for 2-consecutive weeks his power output (W) improved from 225 to 241.5 (a 7.3% increase), while the heart rate reduced by 0.3%.

Subject 3. In another subject, a 39-year old male cyclist, who consumed 1 fl. oz. BID of VMP35 for 2-consecutive weeks his power output (W) improved from 262 to 286.5 (a 9.4% increase), while the heart rate reduced by 1.02%.

Subjects reported that their enthusiasm levels increased significantly. No adverse events were reported by these well-trained athletic cyclists.

Individual Case Study Reports. All case study reports obtained necessary permission from both the patients and supervised physicians.

### EXAMPLE 8

Case Study: An anemic stroke patient. A 56-year old Caucasian male stroke patient (Norwich, N.Y.) suffering from cerebral ischemia and anemia with an extremely low hemoglobin (Hb) level of 2.8 gm/dL consumed (4 fl. oz of VMP35/day) over a period of six consecutive months. Following VMP35 supplementation over a period of six consecutive months Hb levels increased to 15.6 g/dL, while significant increases were observed in other parameters including RBCs to 5.01, Hematocrit to 45.9, Platelet Count to 202, and RDW to 12.9. An evaluation of brain MRI exhibited no signs of brain infarction, and a magnetic resonance angiogram (MRA) showed no signs of occlusion or hemodynamically significant stenosis of major intercranial arteries. These results strengthen that VMP35 can significantly enhance hematological characteristics.

### EXAMPLE 9

Case Study: A motorcycle accident victim. A 33-year-old male subject (Lederach, PA) encountered a motorcycle accident on Oct. 10, 2018, and admitted to Jefferson Hospital with serious life-threatening injuries such as profuse bleeding between lungs and chest wall, collapsed lung, flail chest, fracture in the left acetabulum, and weakness. Clear signs of blood loss-induced anemia were evident from the hematological counts including hematocrit, and hemoglobin levels, and platelet, RBC, and white blood cell counts. Subject received 5-pint plasma infusion within a short span of the accident and undergone extensive surgical procedures. Subject started consuming 6 ounces of VMP35 per day from Oct 13 until Oct 25, and on the same day the subject was released from hospital and continued taking 4 ounces of VMP35 per day until Jan. 31, 2019, and later continued taking a maintenance dose of 2 ounces/day. Along with physical therapy continued although the recovery process was extremely slow and walking only with assistance. It was predicted that the subject will not be able to move appropriately until the late of Spring of 2019.

However, VMP35 significantly accelerated recovery far beyond medical predictions for regaining vitality and optimal functional capabilities. Moreover, both hematocrit and hemoglobin levels remarkably improved including extensive repair of damaged blood vessels and injured tissues. Furthermore, platelet count was normalized.

Conclusions and discussion.

Both people with serious health problems and extreme athletes have in common a need for increased nutritional support to provide nourishment for greater than routine health maintenance. Food sources for the masses include those provided by conventional agribusiness practices (i.e. using chemical fertilizers, pesticides, herbicides, fungicides, growth enhancers, GMO, gassing, irradiation, coloring agents, etc.), food processing (including blanching, preservatives, flavor enhancers, functional food additives, food colorings, etc.), food distributors, snack food products, and fast food outlets. Food stuffs from these sources are not only generally inadequate to meet the special and increased metabolic needs of these populations but are to some extent implicated as a cause of nutritional inadequacies and chemical/toxic insults underlying the shortfalls in both health and enhanced physical performance needs. Dietary supplementation is becoming increasingly commonplace to augment the aforementioned dietary practices and meet nutrition requirements in order to achieve even the minimal functional competence of human biology. It is practically mandatory that both people with chronic disorders and people who engage in more advanced and/or extreme athletic activities increase their nutritional resources through consuming various dietary supplements. The primary etiological factor underlying chronic degenerative diseases (CDD) is the increase in anaerobic events and pathologies; i.e. the inability to effectively use oxygen and water, and therefore nutrients, for cellular energy production, management, and waste removal. Anaerobic pathologies are the consequence of an overburdened pH buffering capability and generate a significant increase in reactive oxygen species (ROS). Given this, in addition to making healthier food choices, supplementation should include ingredients/products that restore aerobic metabolic events, minimizing free radical generation, and provide additional antioxidants to neutralize ROS as well. Clinical research, case studies, and concept validation pilot studies have demonstrated that a WADA compliant iron-free liquid VMP35 dietary supplement supplies an abundant reservoir of buffers to restore aerobic metabolism by restoring iron-dependent hemoglobin to RBCs, bolstering neutrophils in the blood (immune support), and significantly improving performance output in a diverse range of extreme athletes. The VMP35 provides a highly bioavailable source of vitamins, macro and trace minerals, ions, phospholipids, botanicals containing a wide range of evidence-based flavonoids, stilbenes, alkaloids, quinones, phytosaccharides, glycosides, sesquiterpenes, coumarins, polyacetylenes, carotenoids, etc.

Overall, above data strengthen the antioxidant and physiological benefits of structurally-diverse phytonutrients in the bioflavonoid-enriched VMP35 to achieve overall health maintenance for metabolic competence and athletic performance.

In an embodiment, the dosage of the Prodovite® VMP35 formulation may be in water in 1 fl. oz. per day, straight or mixed into or diluted with 1 to 6 fl. oz. of water. Alternatively, in the event of extraordinary health demands, such as extreme athletic endeavors or other strenuous physical activities, about 6 fl. oz. of the formulation may be divided into one or more doses/day or optionally mixed into or diluted with 1 to 6 oz. of water and sipped and swished in the mouth before swallowing.

Discussion concerning treatment of anemia.

Anemia is an alarming health condition of having lower-than-normal red blood cell counts or when red blood cells do not have adequate hemoglobin and/or enough oxygen-rich blood. WHO reported in 2008 that anemia affects 24.8% of the world's population (1.62 billion people) including 47.4% preschoolers, 25.4% school-age children, 41.8% pregnant women, 30.2% non-pregnant women, 12.7% men, and 23.9% of the elderly. See, e.g., R. Stauder, et al., Anemia at older age: etiologies, clinical implications, and management, 131 BLOOD 505 (2018), incorporated by reference herein in its entirety. In 2010, the global prevalence of anemia was estimated to be 32.9%, and, extrapolated in terms of years lived with a disability, the total cumulative amount of time that people were disabled equaled 68.4 million years. See, e.g., N. J. Kassebaum, The Global Burden of Anemia, 30 HEMATOLOGY/ONCOLOGY CLINICS N. AM. 247 (2016); N. J. Kassebaum, et al., A systematic analysis of global anemia burden from 1990 to 2010, 123 BLOOD 615 (2014); B. de Benoist, et al., Worldwide prevalence of anemia 1993-2005: WHO global database on anemia, WORLD HEALTH ORGANIZATION (last visited Dec. 20, 2018), http://apps.who.int/iris/bitstream/handle/10665/43894/9789241596657_eng.pdf;jsessionid=031F 07AE771D458E41369447BD86FE63?sequence=1; S. R. Pasricha, Anemia: a comprehensive global estimate Comment on Kassebaum et al., 123 BLOOD 611 (2014); each of which is incorporated by reference herein in its entirety. Also, anemia is closely associated with, and a precursor of, many disease pathologies, including chronic inflammation, chronic kidney disease, gastrointestinal and gynecological malignancies, and autoimmune disorders; most of these disorders are preceded by an array of dysregulated metabolic events. See, e.g., NAT'L INST. OF DIABETES & DIGESTIVE & KIDNEY DISEASES (NIDDK), Anemia of Inflammation or Chronic Disease (last visited Dec. 20, 2018), https://www.niddk.nih.gov/health-information/blood-diseases/anemia-inflammation-chronic-disease, incorporated by reference herein in its entirety. According to the American Society of Hematology, and the National Heart, Lung, and Blood Institute (“NHLBI”), from the existing perspectives and knowledge, anemia affects more than 3 million Americans. See, e.g., AM. SOC'Y OF HEMATOLOGY, http://www.hematology.org/Patients/Anemia/ (last visited Dec. 20, 2018), incorporated by reference herein in its entirety. The information presented herein suggests that this number may be grossly understated.

Anemia is prevalent in diverse disease pathologies, most of which are precedent and characterized by an increase in anaerobic metabolic events. Therefore, it seems logical and necessary to reevaluate the etiology of anemic disorders. See, e.g., E. Beutler, Red cell enzyme defects, 4 HEMATOLOGIC PATHOLOGY 103 (1990); W. N. Valentine, et al., Hemolytic anemias and erythrocyte enzymopathies, 103 ANNALS OF INTERNAL MEDICINE 245 (1985); A. Bile, et al., Sickle cell trait in Ivory Coast athletic throw and jump champions, 1956-1995, 19 INT'L J. SPORTS MEDICINE 215 (1998); L. Luzzatto, Hemolytic anemias due to enzyme deficiencies, 43 SCHWEIZ MED WOCHENSCHR. SUPPL. 103 (1991); each of which is incorporated by reference herein in its entirety. Optimal health is the result of the body's ability to successfully maintain the ideal, biological environment for optimal cellular functioning. Aerobic cellular events are important for human life. See, e.g., L. Luzzato, 1991; T. Yoshida & S. S. Shevkoplyas, Anaerobic storage of red blood cells, 8 BLOOD TRANSFUSION 220 (2010); K. A. Brown, Erythrocyte Metabolism and Enzyme Defects, 27 LAB. MEDICINE 329 (1996); each of which is incorporated by reference herein in its entirety. Maintaining a highly efficient pH buffering system—a pH between 7.35 and 7.45—is ideal for maintaining optimal oxygenation of the blood in addition to many other biological processes. See, e.g., L. L. Hamm, et al., Acid-Base Homeostasis, 10 CLINICAL J. AM. SOC'Y NEPHROLOGY 2232 (2015); P. Reddy & A. D. Mooradian, Clinical utility of anion gap in deciphering acid-base disorders, 63 INT'L J. CLINICAL PRACTICE 1516 (2009); each of which is incorporated by reference herein in its entirety. Optimizing the ideal pH in the blood is the result of a homeostasis of acid and alkaline pH buffers. A pH of the blood below 7.35 is symptomatic of acidemia, while a pH above 7.45 is symptomatic of alkalemia, and a pH of 7.40 is typically ideal. See, e.g., K. A. Brown, 1996; L. L. Hamm, et al., 2015; P. Reddy & A. D. Mooradian, 2009; C. P. Thomas & K. Hamawi, What is the role of acidemia and alkalemia in the pathogenesis of metabolic acidosis?, MEDSCAPE.COM, https://www.medscape.com/answers/242975-154551/what-is-the-role-of-acidemia-and-alkalemia-in-the-pathogenesis-of-metabolic-acidosis (last visited Dec. 20, 2018); Erin Hopkins & Sandeep Sharma, Physiology, Acid Base Balance, STATPEARLS, https://www.ncbi.nlm.nih.gov/books/NBK507807/ (last visited Dec. 20, 2018); each of which is incorporated by reference herein in its entirety. Due to the importance of sustaining a pH level in the narrow-specified range, the human body exerts a compensatory mechanism to induce a homeostatic counterbalance.

An array of chronic diseases can be characterized by an increased inability to effectively utilize oxygen, resulting in anaerobic metabolism and lactate accumulation. See, e.g., B. Phypers & T. Pierce, Lactate physiology in health and disease, 6 CONTINUING EDUCATION IN ANAESTHESIA CRITICAL CARE & PAIN 128 (2006), incorporated by reference herein in its entirety. Impairment of oxidative pathways during lactate production results in a net gain of protons with increasing cellular acid burden, thereby decreasing the pH of the blood.

As discussed above, the etiology of Chronic Anemia Syndrome (CAS) includes a progressive inability to effectively use cellular oxygen, inducing progressive acidemia, a metabolic shift toward cellular anaerobic glycolysis, and a compensatory expenditure of alkalinizing histidine molecules from the heme protein of deconjugated hemoglobin, which releases iron. Iron is taken out of circulation and accumulates in the liver, bone marrow, and other organs, which appears to be iron deficiency anemia (IDA), but can result in dangerous accumulations of iron. In addition to iron accumulating in certain organs, the consequences of an increasing anaerobic/acidic environment, especially in the blood, can manifest in a number of ways, in various tissues, and produce a wide range of symptoms and pathological manifestations, all of which are preceded by CAS, including, but not limited to, chronic and acute infections, flukes, vaso-occlusive incidences, cardiovascular disease (“CVD”), strokes, kidney disease, cancers, diabetes, tuberculosis, HIV, endocarditis, osteomyelitis, and inflammatory bowel diseases such as Crohn's disease.

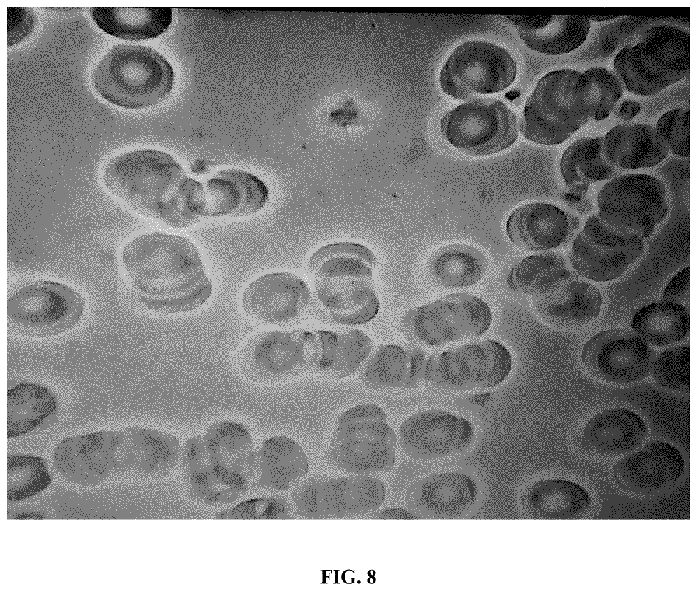

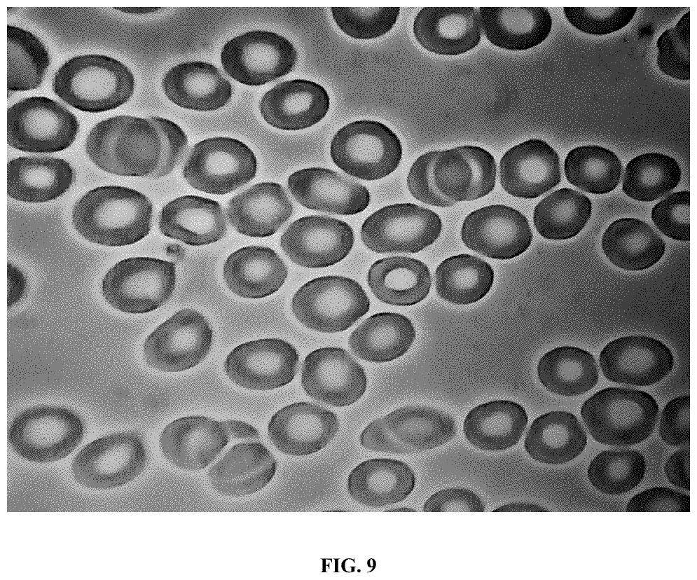

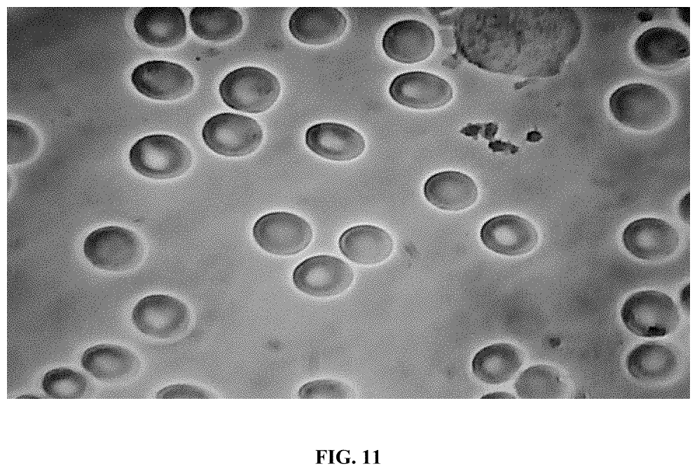

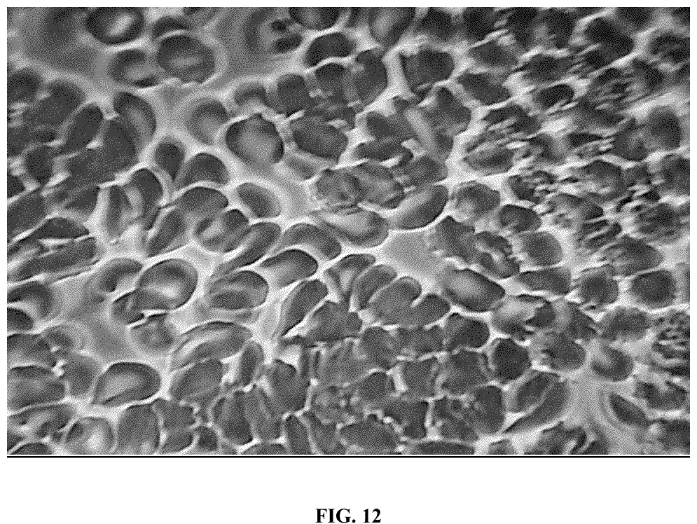

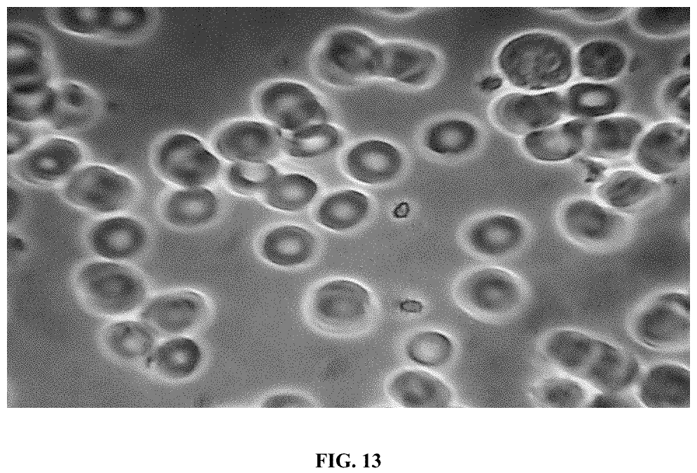

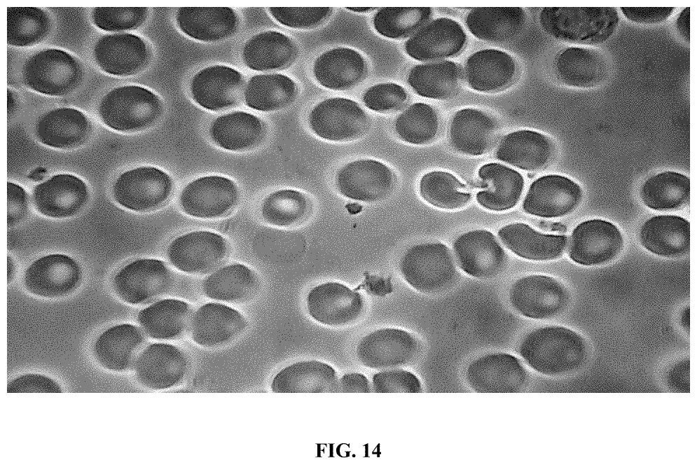

Chronic Anemia Syndrome (CAS) is intricately associated with hemoglobin (“Hb”), which is usually checked in a complete blood count (“CBC”). Structurally, Hb is composed of four chains, two α-globulin and two β-globulin chains, and each chain is known as heme, which contains iron and is responsible for transporting oxygen in the bloodstream. See, e.g., INFORMATION CTR. FOR SICKLE CELL & THALASSEMIC DISORDERS, An Overview of Hemoglobin, http://sickle.bwh.harvard.edu/hemoglobin.html (last visited Dec. 21, 2018); G. Weiss & L. T. Goodnough, Anemia of chronic disease, 352 NEW ENGLAND J. MEDICINE 1011 (2005); each of which is incorporated by reference herein in its entirety. Basically, Hb is a protein molecule in red blood cells that transports oxygen from the lungs and delivers it to the peripheral tissues to maintain the viability of cells and returns CO₂ from the organs and tissues back to the lungs. An upsurge of CO₂ acts as an acidifying buffer. CO₂ interacts with water to form carbonic acid and results in a decrease of blood pH. Also, the pigment in Hb in responsible for its red color. The normal, average range of Hb is between about 14 and about 18 g/dL for an adult male, and between about 12 and about 16 g/dL for an adult female. Abnormal levels or deformation of Hb can lead to serious diseases or consequences, while low Hb level is referred to as anemia. Hb is responsible for the shape of the red blood cells, which look like donuts with thin centers, instead of a hole, and generally spherical, after taking the indentation into account, when viewed from the top. However, for dysfunctional or deformation of Hb, which is predominant during anemia, the shape of red blood cells becomes abnormal. (See, e.g., FIGS. 7-14).

Anemia has been demonstrated to be caused by factors that interfere either with the compromised Hb level or the absence of adequate red blood cells or oxygen deprivation. There are six underlying causes: (i) loss of red blood cells due to bleeding, as in hemorrhagic anemia; (ii) a lack of production of red blood cells in the bone marrow; (iii) hemolysis or breakdown or deformation of red blood cells in the blood stream; (iv) nutritional deficiency or inadequate intake of iron, folic acid, or vitamin B12; (v) kidney disease; and (vi) genetic predisposition. Compromised Hb proteins ultimately release their load of oxygen. See, e.g., V. G. Sankaran & M. J. Weiss, Anemia: Progress in molecular mechanisms and therapy, 21 NATURE MEDICINE 221 (2015); N. Touret, et al., Molecular and cellular mechanisms underlying iron transport deficiency in microcytic anemia, 104 BLOOD 1526 (2004); each of which is incorporated by reference herein in its entirety. In conjunction, increased acid burden in the blood can be characterized by an increase in the CO₂ load in the blood gas, and this effect induces a secondary role in ionizing minerals in the kidney. Consequently, anaerobic disorders promote kidney problems. See, e.g., Mitchell H. Rosner & Mark A. Perazella, Acute Kidney Injury in Patients With Cancer, 32 ONCOLOGY 351 (2018); Mitchell H. Rosner & Mark A. Perazella, Acute Kidney Injury in Patients With Cancer, 376 NEW ENGLAND J. MEDICINE 1770 (2017); each of which is incorporated by reference herein in its entirety.

Hb is divided into two types: (a) oxyhemoglobin, the red, oxygen-carrying form; and (b) deoxyhemoglobin, the blue/purple deoxygenated, or reduced, form. Hb is also expected to perform an important task as a pH-buffering agent, to regulate and optimize cellular oxygen utilization. This role is primarily dependent upon hemoglobin's histidine of its heme groups. See, e.g., K. Nishikura, Identification of histidine-122a in human haemoglobin as one of the unknown alkaline Bohr groups by hydrogen-tritium exchange, 173 BIOCHEMICAL J. 651 (1978), incorporated by reference herein in its entirety. Under pH regulatory distress (i.e., increasing acid burden, and significantly increasing alkalinizing buffers), heme groups are broken down to release histidine. See, e.g., H. S. Jacob & K. H. Winterhalter, The role of hemoglobin heme loss in Heinz body formation: studies with a partially heme-deficient hemoglobin and with genetically unstable hemoglobins, 49 J. CLINICAL INVESTIGATION 2008 (1970); D. K. Miller & C. P. Rhoads, The experimental production of loss of hematopoietic elements of the gastric secretion and of the liver in swine with achlorhydria and anemia, 14 J. CLINICAL INVESTIGATION 153 (1935); each of which is incorporated by reference herein in its entirety. Iron is released simultaneously with the histidine, and is rapidly sent to other organs including the liver, bone marrow, heart, pancreas, and intestinal mucosa. During the transport, iron remains in the blood for a very short time. However, in more extreme cases, the iron accumulates in vital organs, which contributes to the development of several diseases including hepatic cirrhosis, hepatocarcinoma, cardiac cirrhosis, diabetes mellitus, osteoarthritis, osteoporosis, and non-genetic secondary hemochromatosis (i.e., “iron-overload anemia”), because blood iron levels can be initially low with accumulated tissue levels being too high. See, e.g., M. Simäo, et al., Iron-enriched diet contributes to early onset of osteoporotic phenotype in a mouse model of hereditary hemochromatosis, 13 PLOS ONE e0207441 (2018), incorporated by reference herein in its entirety. This is a major reason why blood tests can be inaccurate in determining IDA. However, prolonged accumulation of iron in the organs can lead to critical saturation in the tissues and iron overload, with a downregulation of receptor sensitivity, and once again elevate blood iron levels. As such, increased saturation of transferrin and high iron in plasma (i.e., extracellular concentrations) are hallmarks of hemochromatosis forms as well. Tests to evaluate for this condition include, but are not limited to, serum transferrin saturation, serum ferritin, liver function tests, MRI, and liver tissue biopsy. See, e.g., P. C. Adams, Epidemiology and diagnostic testing for hemochromatosis and iron overload, 37 (Suppl. 1) INT'L J. LAB. HEMATOLOGY 25 (2015); H. Zoller & B. Henninger, Pathogenesis, Diagnosis and Treatment of Hemochromatosis, 34 DIGESTIVE DISEASES 364 (2016); each of which is incorporated by reference herein in its entirety. It is not uncommon that elevations in one or all of these blood tests for iron can be found in other disorders as well, owing to the prevalence of the anaerobic/acidic bio-environment underlying chronic disease pathologies.

Rather than considering anemia as a consequence of disease, it should not be recognized as an etiological antecedent, a primary underlying cause to all of the clinical anaerobic pathologies and disorders mentioned above. More accurate characterizations are needed to indicate two distinct types of anemia that are not caused by gene mutations. The first such type of anemia is “hemorrhagic anemia,” evidenced by either acute or chronic hemorrhaging, resulting in a detectable and substantial loss of blood, and its consequential alterations in all blood parameters, including reduced iron. The cause of the hemorrhage could be, for example, traumatic injury. The second such type of anemia is “Chronic Anemia Syndrome,” representing a wide variety of anemic pathologies resulting from a deficiency in alkaline buffers, inducing the expenditure of histidine from heme groups, consequently causing the anemic conditions. This defensive mechanism is apparently a precursor to numerous other anaerobic pathologies. It was hypothesized that highly absorbable and comprehensive supplemental nutrient repletion from a VMP35 MNC composition would provide the buffers necessary to halt hemoglobin expenditure, effectively puling excess accumulated iron from other tissue storage depots, enabling rapid reconstitution of RBC hemoglobin.

To test the hypothesis, a pilot clinical study was conducted, and the study demonstrated that a VMP35 non-iron-containing liquid supplement, encapsulated in a Prodosome, rapidly improved morphological, hematological, and rheological properties of live human blood. Moreover, this study exhibited that sufficient nutritional and phytochemical resources were available from the VMP35 MNC to provide adequate buffering to restore intracellular red blood cell hemoglobin within 5 minutes of intake and that were sustained for at least 30 minutes post intake. In addition, properties of white blood cells, including neutrophils, significantly improved. Further, the case study report is very encouraging, further strengthening the findings of Prodovite® VMP35. Taken together, VMP35, a non-iron-based nutraceutical supplement, has surprisingly and unexpectedly been shown to serve as a novel therapeutic intervention in the restoration of hemoglobin in red blood cells and to reverse the progression of anemic pathologies.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value either above or below the stated value in a range of approximately ±5%; in other embodiments, the values may range in value either above or below the stated value in a range of approximately ±2%; in other embodiments, the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A method for treating or preventing chronic anemia syndrome in a human subject in need of such treatment, comprising the steps of:
(a) providing a formulation comprising one or more vitamins or multivitamins, one or more macro or trace minerals, one or more botanical nutrients or phytonutrients, and a multilamellar clustoidal phospholipid vehicle, the multilamellar clustoidal phospholipid vehicle comprising:
a solvent,
phosphatidylcholine of at least 70% purity, and
an ionic mineral composition essentially free of iron;
(b) administering the formulation in an effective amount to treat the human subject; and
(c) measuring at least one blood parameter in the human subject;
wherein red blood cell (RBC) morphology and/or hemoglobin saturation are improved.

2. The method of claim 1, wherein the composition is administered orally or transmucosally.

3. The method of claim 1, wherein the phytonutrients are selected from the group consisting of Astragalus Root extract, Fo ti Root extract, Green tea Leaf extract, Chamomile Flower extract, Citrus bioflavonoids (from Rose hips extract), Eleuthero Root extract, Hawthorne berry extract, Gotu kola Leaf extract, Ginger Root extract, Elderberry extract, Aloe vera, and White pine cone extract.

4. The method of claim 1, wherein the vitamins are selected from one or more Vitamin B Complex, Vitamin A, Vitamin C, Vitamin D3, and Vitamin E.

5. The method of claim 1, wherein the Hemoglobin (Hb) level is increased and normalized.

6. The method of claim 5, wherein the human subject is male and the Hemoglobin (Hb) level is between about 14 and about 18 g/dL in blood.

7. The method of claim 5, wherein the human subject is female and the Hemoglobin (Hb) level is between about 12 and about 16 g/dL in blood.

8. The method of claim 1, wherein the RBC morphology is improved as evidenced by one or more of spherical formation, reduced hypochromicity, improved hydration, reduction of aggregation, improved ability to flow, and reduced viscosity.

9. The method of claim 8, wherein the RBC morphology in the blood of the human improves within from about 5 minutes to about 60 minutes after administering the formulation.

10. The method of claim 8, wherein the RBC morphology in the blood of the human improves within from about 5 minutes to about 30 minutes after administering the formulation.

11. The method of claim 8, wherein the dosage of the formulation is from about 1 fl. oz. per day in water to about 6 fl. oz. per day in water, optionally mixed in with additional 1 to 6 fl. oz. water.

12. The method of claim 1, wherein oxygen saturation is increased as measured in blood.

13. A method for improving physical performance in a human subject, comprising the steps of:
(a) providing a formulation comprising one or more vitamins or multivitamins, one or more macro or trace minerals, one or more botanical nutrients or phytonutrients, and a multilamellar clustoidal phospholipid vehicle, the multilamellar clustoidal phospholipid vehicle comprising:
a solvent,
phosphatidylcholine of at least 70% purity, and
an ionic mineral composition essentially free of iron;
(b) administering the formulation in an effective amount to treat the human subject; and
(c) measuring at least one blood parameter in the human subject;
wherein red blood cell (RBC) morphology and/or hemoglobin saturation are improved.

14. The method of claim 13, wherein the composition is administered orally or transmucosally.

15. The method of claim 13, wherein the phytonutrients are selected from the group consisting of Astragalus Root extract, Fo ti Root extract, Green tea Leaf extract, Chamomile Flower extract, Citrus bioflavonoids (from Rose hips extract), Eleuthero Root extract, Hawthorne berry extract, Gotu kola Leaf extract, Ginger Root extract, Elderberry extract, Aloe vera, and White pine cone extract.

16. The method of claim 13, wherein the vitamins are selected from one or more Vitamin B Complex, Vitamin A, Vitamin C, Vitamin D3, and Vitamin E.

17. The method of claim 13, wherein the Hemoglobin (Hb) level is increased and normalized.

18. The method of claim 17, wherein the human subject is male and the Hemoglobin (Hb) level is between about 14 and about 18 g/dL in blood.

19. The method of claim 17, wherein the human subject is female and the Hemoglobin (Hb) level is between about 12 and about 16 g/dL in blood.

20. The method of claim 13, wherein the RBC morphology is improved as evidenced by one or more of spherical formation, reduced hypochromicity, improved hydration, reduction of aggregation, improved ability to flow, and reduced viscosity.

21. The method of claim 20, wherein the RBC morphology in the blood of the human improves after about 5 minutes to about 60 minutes after administering the formulation.

22. The method of claim 20, wherein the RBC morphology in the blood of the human improves after about 5 minutes to about 30 minutes after administering the formulation.

23. The method of claim 20, wherein the dosage of the formulation is from about 1 fl. oz. per day in water to about 6 fl. oz. per day in water, optionally mixed in with additional 1 to 6 fl. oz. water.

24. The method of claim 23, wherein the dosage may be provided in several separate doses, each optionally mixed in with additional 1 to 6 fl. oz. water.

25. The method of claim 1, wherein oxygen saturation is increased as measured in blood.
